SEQUENCE LISTING (33 sequences)

SEQ ID NO: 1 - DNA (cDNA), 1060 bp; single-stranded, linear
  mat_peptide 73..924
  sig_peptide 10..72
  CDS 10..924
  hypothetical: NO
  original source strain: DSM 1800
ggatccaagatgcgttcctcccccctcctcccgtccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggcggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatcccctccagcagcaccagctctccggtcaaccagcctaccagcaccagcaccacgtccacctccaccacctcgagcccgccagtccagcctacgactcccagcggctgcactgctgagaggtgggctcagtgcggcggcaatggctggagcggctgcaccacctgcgtcgctggcagcacttgcacgaagattaatgactggtaccatcagtgcctgtagacgcagggcagcttgagggccttactggtggccgcaacgaaatgacactcccaatcactgtattagttcttgtacataatttcgtcatccctccagggattgtcacataaatgcaatgaggaacaatgagtac

SEQ ID NO: 2 - protein, 305 aa
MRSSPLLPSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPSSSTSSPVNQPTSTSTTSTSTTSSPPVQPTTPSGCTAERWAQCGGNGWSGCTTCVAGSTCTKINDWYHQCL

SEQ ID NO: 3 - DNA (cDNA), 1473 bp, Fusarium oxysporum; single-stranded, linear
  CDS 97..1224
  hypothetical: NO
  anti-sense: NO
  original source strain: DSM 2672
gaattcgcggccgctcattcacttcattcattctttagaattacatacactctctttcaaaacagtcactctttaaacaaaacaacttttgcaacaatgcgatcttacactcttctcgccctggccggccctctcgccgtgagtgctgcttctggaagcggtcactctactcgatactgggattgctgcaagccttcttgctcttggagcggaaaggctgctgtcaacgcccctgctttaacttgtgataagaacgacaaccccatttccaacaccaatgctgtcaacggttgtgagggtggtggttctgcttatgcttgcaccaactactctccctgggctgtcaacgatgagcttgcctacggtttcgctgctaccaagatctccggtggctccgaggccagctggtgctgtgcttgctatgctttgaccttcaccactggccccgtcaagggcaagaagatgatcgtccagtccaccaacactggaggtgatctcggcgacaaccacttcgatctcatgatgcccggcggtggtgtcggtatcttcgacggctgcacctctgagttcggcaaggctctcggcggtgcccagtacggcggtatctcctcccgaagcgaatgtgatagctaccccgagcttctcaaggacggttgccactggcgattcgactggttcgagaacgccgacaaccctgacttcacctttgagcaggttcagtgccccaaggctctcctcgacatcagtggatgcaagcgtgatgacgactccagcttccctgccttcaaggttgatacctcggccagcaagccccagccctccagctccgctaagaagaccacctccgctgctgctgccgctcagccccagaagaccaaggattccgctcctgttgtccagaagtcctccaccaagcctgccgctcagcccgagcctactaagcccgccgacaagccccagaccgacaagcctgtcgccaccaagcctgctgctaccaagcccgtccaacctgtcaacaagcccaagacaacccagaaggtccgtggaaccaaaacccgaggaagctgcccggccaagactgacgctaccgccaaggcctccgttgtccctgcttattaccagtgtggtggttccaagtccgcttatcccaacggcaacctcgcttgcgctactggaagcaagtgtgtcaagcagaacgagtactactcccagtgtgtccccaactaaatggtagatccatcggttgtggaagagactatgcgtctcagaagggatcctctcatgagcaggcttgtcattgtatagcatggcatcctggaccaagtgttcgacccttgttgtacatagtatatcttcattgtatatatttagacacatagatagcctcttgtcagcgacaactggctacaaaagacttggcaggcttgttcaatattgacacagtttcctccataaaaaaaaaaaaaaaaaaaa

SEQ ID NO: 4 - protein, 376 aa
MRSYTLLALAGPLAVSAASGSGHSTRYWDCCKPSCSWSGKAAVNAPALTCDKNDNPISNTNAVNGCEGGGSAYACTNYSPWAVNDELAYGFAATKISGGSEASWCCACYALTFTTGPVKGKKMIVQSTNTGGDLGDNHFDLMMPGGGVGIFDGCTSEFGKALGGAQYGGISSRSECDSYPELLKDGCHWRFDWFENADNPDFTFEQVQCPKALLDISGCKRDDDSSFPAFKVDTSASKPQPSSSAKKTTSAAAAAQPQKTKDSAPVVQKSSTKPAAQPEPTKPADKPQTDKPVATKPAATKPVQPVNKPKTTQKVRGTKTRGSCPAKTDATAKASVVPAYYQCGGSKSAYPNGNLACATGSKCVKQNEYYSQCVPN

SEQ ID NO: 5 - DNA (genomic), 27 bp; single-stranded, linear
agctgcggccgcaggccgcggaggcca

SEQ ID NO: 6 - DNA (genomic), 27 bp; single-stranded, linear
agcttggcctccgcggcctgcggccgc

SEQ ID NO: 7 - DNA (genomic), 26 bp; single-stranded, linear
aattcgcggccgcggccatggaggcc

SEQ ID NO: 8 - DNA (genomic), 26 bp; single-stranded, linear
aattggcctccatggccgcggccgcg

SEQ ID NO: 9 - DNA (cDNA), 15 bp; single-stranded, linear
aaygcygacaaaycc

SEQ ID NO: 10 - DNA (cDNA), 20 bp; single-stranded, linear
aacgaygayggnaayttccc

SEQ ID NO: 11 - DNA (cDNA), 20 bp; single-stranded, linear
aaygaytggtaccaycartg

SEQ ID NO: 12 - DNA (cDNA), 26 bp; single-stranded, linear
gcgccagtagcagccgggcttgaggg

SEQ ID NO: 13 - DNA (cDNA), 28 bp; single-stranded, linear
acgtctcaactcggatccaagatgcgtt

SEQ ID NO: 14 - DNA (cDNA), 25 bp; single-stranded, linear
ctcaactctgatcaagatgcgttcc

SEQ ID NO: 15 - DNA (cDNA), 26 bp; single-stranded, linear
tgtcgaccagtaaggccctcaagctg

SEQ ID NO: 16 - RNA (genomic), 30 nt; single-stranded, linear
gacagagcacagaattcactagtgagctct

SEQ ID NO: 17 - DNA (cDNA), 17 bp; single-stranded, linear
tgggaytgytgyaarcc

SEQ ID NO: 18 - DNA (cDNA), 33 bp; single-stranded, linear
agggagaccggaattctgggaytgytgyaarcc

SEQ ID NO: 19 - DNA (cDNA), 17 bp; single-stranded, linear
ccnggnggnggngtngg

SEQ ID NO: 20 - DNA (cDNA), 33 bp; single-stranded, linear
agggagaccggaattcccnggnggnggngtngg

SEQ ID NO: 21 - DNA (cDNA), 17 bp; single-stranded, linear
acnaycatnktyttncc

SEQ ID NO: 22 - DNA (cDNA), 34 bp; single-stranded, linear
gacagagcacagaattcacnaycatnktyttncc

SEQ ID NO: 23 - DNA (cDNA), 24 bp; single-stranded, linear
nggrttrtcngcnkyytyraacca

SEQ ID NO: 24 - DNA (cDNA), 41 bp; single-stranded, linear
gacagagcacagaattcnggrttrtcngcnkyytyraacca

SEQ ID NO: 25 - DNA (cDNA), 42 bp; single-stranded, linear
ggggtagctatcacattcgcttcgggaggagataccgccgta

SEQ ID NO: 26 - DNA (cDNA), 42 bp; single-stranded, linear
cttcttgctcttggagcggaaaggctgctgtcaacgcccctg

SEQ ID NO: 27 - DNA (cDNA), 18 bp; single-stranded, linear
tgtacgcatgtaacatta

SEQ ID NO: 28 - DNA (cDNA), 18 bp; single-stranded, linear
ctgcacaatatttcaagc

SEQ ID NO: 29 - DNA (cDNA), 42 bp; single-stranded, linear; identical to SEQ ID NO: 25

SEQ ID NO: 30 - DNA (cDNA), 42 bp; single-stranded, linear; identical to SEQ ID NO: 26

SEQ ID NO: 31 - DNA (cDNA), 18 bp; single-stranded, linear
agcttctcaaggacggtt

SEQ ID NO: 32 - DNA (cDNA), 18 bp; single-stranded, linear
aacaagggtcgaacactt

SEQ ID NO: 33 - DNA (cDNA), 17 bp; single-stranded, linear
ccagaagaccaaggatt

(19) United States
(12) Patent Application Publication
(10) Pub. No.: US 2003/0119167 A1
(43) Pub. Date: Jun. 26, 2003

(54) Cellulase preparation comprising an endoglucanase enzyme

(75) Inventors: Grethe Rasmussen, Copenhagen (DK); Jan Moller Mikkelsen, Gentofte (DK); Martin Schulein, Copenhagen (DK); Shamkant Anant Patkar, Lyngby (DK); Fred Hagen, Seattle, WA (US); Carsten Mailand Hjort, Roskilde (DK); Sven Hastrup, Copenhagen (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/138,870

(22) Filed: May 3, 2002

Related U.S. Application Data
(63) Continuation of application No. 09/735,787, filed on Dec. 13, 2000.
Application No. 09/735,787 is a continuation of application No. 09/189,028, filed on Nov. 10, 1998, now Pat. No. 6,423,524.
Application No. 09/189,028 is a division of application No. 08/389,423, filed on Feb. 14, 1995, now Pat. No. 5,948,672.
Application No. 08/389,423 is a continuation of application No. 07/946,489, filed on Nov. 25, 1992, now abandoned.
Application No. 07/946,489 is a 371 of international application No. PCT/DK91/00123, filed on May 8, 1991.

(30) Foreign Application Priority Data
May 9, 1990 (DK) 1159/90
Apr. 22, 1991 (DK) 736/91

(51) Int. Cl. C12N 9/42; C07H 21/04; C12P 21/02; C12N 1/21; C12N 15/74

(52) U.S. Cl. 435/209; 435/69.1; 435/320.1; 435/252.3; 536/23.2

(57) ABSTRACT

The present invention relates to cellulase preparations consisting essentially of a homogeneous endoglucanase component which is immunoreactive with an antibody raised against a highly purified ˜43 kD endoglucanase derived from Humicola insolens, DSM 1800, or which is homogenous to said ˜43 kD endoglucanase, may be employed in the treatment cellulose-containing fabrics for harshness reduction or color clarification or to provide a localized variation in the color of such fabrics, or it may be employed in the treatment of paper pulp.

### CROSS-REFERENCE TO RELATED APPLICATIONS

This application is a continuation of U.S. application Ser. No. 09/735,787 filed Dec. 13, 2000, which is a continuation of U.S. application Ser. No. 09/189,028 filed Nov. 10, 1998, which is a division of U.S. application Ser. No. 08/389,423 filed Feb. 14, 1995, which is a continuation of U.S. application Ser. No. 07/946,489 filed Nov. 25, 1992, which is a 35 U.S.C. 371 application of PCT/DK91/00123 filed May 8, 1991, and claims priority under 35 U.S.C. 119 of Danish application nos. 1159/90 and 736/91 filed May 9, 1990 and Apr. 22, 1991, respectively, the contents of which are fully incorporated herein by reference.

### FIELD OF INVENTION

The present invention concerns a cellulase preparation comprising a single-component endoglucanase, a detergent additive comprising the cellulase preparation, a detergent composition containing the cellulase preparation as well as methods of treating cellulose-containing fabrics with the cellulase preparation.

### BACKGROUND OF THE INVENTION

It is well known in the art that repeated washing of cotton-containing fabrics generally causes a pronounced, unpleasant harshness in the fabric, and several methods for overcoming this problem have previously been suggested in the art. For example GB 1,368,599 of Unilever Ltd. teaches the use of cellulytic enzymes for reducing the harshness of cotton-containing fabrics. Also, U.S. Pat. No. 4,435,307 (of Novo Industri A/S) teaches the use of a cellulytic enzyme derived from Humicola insolens as well as a fraction thereof, designated ACₓI, as a harshness reducing detergent additive. Other uses of cellulytic enzymes mentioned in the art involve soil removal from and color clarification of fabric (cf. for instance EP 220 016), providing increasing water absorption (JP-B-52-48236) and providing a localized variation in color to give the treated fabrics a “stone-washed” appearance (EP 307,564). Cellulytic enzymes may furthermore be used in the brewing industry for the degradation of beta-glucans, in the baking industry for improving the properties of flour, in paper pulp processing for removing the non-crystalline parts of cellulose, thus increasing the proportion of crystalline cellulose in the pulp, and for improving the drainage properties of pulp, and in animal feed for improving the digestibility of glucans.

The practical exploitation of cellulytic enzymes has, to some extent, been set back by the nature of the known cellulase preparations which are often complex mixtures. It is difficult to optimize the production of multiple enzyme systems and thus to implement industrial cost-effective production of cellulytic enzymes, and their actual use has been hampered by difficulties arising from the need to apply rather large quantities of the cellulytic enzymes to achieve the desired effect on cellulosic fabrics.

The drawbacks of previously suggested cellulase preparations may be remedied by using preparations comprising a higher amount of endoglucanases. A cellulase preparation enriched in endoglucanase activity is disclosed in WO 89/00069.

### SUMMARY OF THE INVENTION

A single endoglucanase component has now been isolated which exhibits favourable activity levels relative to cellulose-containing materials.

Accordingly, the present invention relates to a cellulase preparation consisting essentially of a homogeneous endoglucanase component which is immunoreactive with an antibody raised against a highly purified ˜43 kD endoglucanase derived from Humicola insolens, DSM 1800, or which is homologous to said ˜43 kD endoglucanase.

The finding that this particular endoglucanase component of cellulase is advantageous for the treatment of cellulose-containing materials is of considerable practical significance: it permits a cost-effective production of the cellulase, e.g. by employing recombinant DNA techniques for producing the active component, and makes the actual effective application of the enzyme feasible in that a smaller quantity of the cellulase preparation is requested to produce the desired effect on cellulosic materials.

### BRIEF DESCRIPTION OF THE DRAWINGS

FIG. 1 illustrates the creation of the Aspergillus expression vector pToC 68.

FIG. 2 illustrates the cloning strategy for the cloning of the full-length cDNA molecule encoding the 43 kD endoglucanase from Humicola insolens strain DSM 1800.

### DETAILED DISCLOSURE OF THE INVENTION

The cellulase preparation of the invention is advantageously one in which the endoglucanase component exhibits a CMC-endoase activity of at least about 50 CMC-endoase units per mg of total protein.

In the present context, the term “CMC-endoase activity” refers to the endoglucanase activity of the endoglucanase component in terms of its ability to degrade cellulose to glucose, cellobiose and triose, as determined by a viscosity decrease of a solution of carboxymethyl cellulose (CMC) after incubation with the cellulase preparation of the invention, as described in detail below.

Preferred cellulase preparations of the invention are those in which the endoglucanase component exhibits a CMC-endoase activity of at least about 60, in particular at least about 90, CMC-endoase units per mg of total protein. In particular, a preferred endoglucanase component exhibits a CMC-endoase activity of at least 100 CMC-endoase units per mg of total protein.

The CMC-endoase (endoglucanase) activity can be determined from the viscosity decrease of CMC, as follows:

A substrate solution is prepared, containing 35 g/l CMC (Hercules 7 LFD) in 0.1 M tris buffer at pH 9.0. The enzyme sample to be analyzed is dissolved in the same buffer.

Ten ml substrate solution and 0.5 ml enzyme solution are mixed and transferred to a viscosimeter (e.g. Haake VT 181, NV sensor, 181 rpm), thermostated at 40° C.

Viscosity readings are taken as soon as possible after mixing and again 30 minutes later. The amount of enzyme that reduces the viscosity to one half under these conditions is defined as 1 unit of CMC-endoase activity.

SDS polyacrylamide gel electrophoresis (SDS-PAGE) and isoelectric focusing with marker proteins in a manner known to persons skilled in the art were used to determine the molecular weight and isoelectric point (pI), respectively, of the endoglucanase component in the cellulase preparation of the invention. In this way, the molecular weight of a specific endoglucanase component was determined to be 43 kD. The isoelectric point of this endoglucanase was determined to be about 5.1. The immunochemical characterization of the endoglucanase was carried out substantially as described in WO 89/00069, establishing that the endoglucanase is immunoreactive with an antibody raised against highly purified ˜43 kD endoglucanase from Humicola insolens, DSM 1800. The cellobiohydrolase activity may be defined as the activity towards cellobiose p-nitrophenyl. The activity is determined as micromoles nitrophenyl released per minute at 37° C. and pH 7.0. The present endoglucanase component was found to have essentially no cellobiohydrolase activity.

The endoglucanase component in the cellulase preparation of the invention has initially been isolated by extensive purification procedures, i.a. involving reverse phase HPLC purification of a crude H. insolens cellulase mixture according to U.S. Pat. No. 4,435,307 (cf. Example 1 below). This procedure has surprisingly resulted in the isolation of a ˜43 kD endoglucanase as a single component with unexpectedly favourable properties due to a surprisingly high endoglucanase activity.

In another aspect, the present invention relates to an enzyme exhibiting endoglucanase activity (in the following referred to as an “endoglucanase enzyme”), which enzyme has the amino acid sequence shown in SEQ ID NO: 2, or a homologue thereof exhibiting endoglucanase activity. In the present context, the term “homologue” is intended to indicate a polypeptide encoded by DNA which hybridizes to the same probe as the DNA coding for the endoglucanase enzyme with this amino acid sequence under certain specified conditions (such as presoaking in 5× SSC and prehybridizing for 1 h at ˜40° C. in a solution of 20% formamide, 5× Denhardt's solution, 50 mM sodium phosphate, pH 6.8, and 50 micrograms of denatured sonicated calf thymus DNA, followed by hybridization in the same solution supplemented with 100 micro-M ATP for 18 h at ˜40° C.). The term is intended to include derivatives of the aforementioned sequence obtained by addition of one or more amino acid residues to either or both the C- and N-terminal of the native sequence, substitution of one or more amino acid residues at one or more sites in the native sequence, deletion of one or more amino acid residues at either or both ends of the native amino acid sequence or at one or more sites within the native sequence, or insertion of one or more amino acid residues at one or more sites in the native sequence.

The endoglucanase enzyme of the invention may be one producible by species of Humicola such as Humicola insolens e.g strain DSM 1800, deposited on Oct. 1, 1981 at the Deutsche Sammlung von Mikroorganismen, Mascheroder Weg 1B, D-3300 Braunschweig, FRG, in accordance with the provisions of the Budapest Treaty on the International Recognition of the Deposit of Microorganisms for the Purposes of Patent Procedure (the Budapest Treaty).

In a further aspect, the present invention relates to an endoglucanase enzyme which has the amino acid sequence shown in SEQ ID NO: 4, or a homologue thereof (as defined above) exhibiting endoglucanase activity. Said endoglucanase enzyme may be one producible by a species of Fusarium, such as Fusarium oxysporum, e.g. strain DSM 2672, deposited on Jun. 6, 1983 at the Deutsche Sammlung von Mikroorganismen, Mascheroder Weg 1B, D-3300 Braunschweig, FRG, in accordance with the provisions of the Budapest Treaty.

Furthermore, it is contemplated that homologous endoglucanases may be derived from other microorganisms producing cellulolytic enzymes, e.g. species of Trichoderma, Myceliophthora, Phanerochaete, Schizophyllum, Penicillium, Aspergillus, and Geotricum.

The present invention also relates to a DNA construct comprising a DNA sequence encoding an endoglucanase enzyme as described above, or a precursor form of the enzyme. In particular, the DNA construct has a DNA sequence as shown in SEQ ID NO: 1 or 3, or a modification thereof. Examples of suitable modifications of the DNA sequence are nucleotide substitutions which do not give rise to another amino acid sequence of the endoglucanase, but which correspond to the codon usage of the host organism into which the DNA construct is introduced or nucleotide substitutions which do give rise to a different amino acid sequence and therefore, possibly, a different protein structure which might give rise to an endoglucanase mutant with different properties than the native enzyme. Other examples of possible modifications are insertion of one or more nucleotides into the sequence, addition of one or more nucleotides at either end of the sequence, or deletion of one or more nucleotides at either end or within the sequence.

The DNA construct of the invention encoding the endoglucanase enzyme may be prepared synthetically by established standard methods, e.g. the phosphoamidite method described by S. L. Beaucage and M. H. Caruthers, Tetrahedron Letters 22, 1981, pp. 1859-1869, or the method described by Matthes et al., EMBO Journal 3, 1984, pp. 801-805. According to the phosphoamidite method, oligonucleotides are synthesized, e.g. in an automatic DNA synthesizer, purified, annealed, ligated and cloned in suitable vectors.

A DNA construct encoding the endoglucanase enzyme or a precursor thereof may, for instance, be isolated by establishing a cDNA or genomic library of a cellulase-producing microorganism, such as Humicola insolens, DSM 1800, and screening for positive clones by conventional procedures such as by hybridization using oligonucleotide probes synthesized on the basis of the full or partial amino acid sequence of the endoglucanase in accordance with standard techniques (cf. Sambrook et al., Molecular Cloning: A Laboratory Manual, 2nd. Ed., Cold Spring Harbor, 1989), or by selecting for clones expressing the appropriate enzyme activity (i.e. CMC-endoase activity as defined above), or by selecting for clones producing a protein which is reactive with an antibody against a native cellulase (endoglucanase).

Finally, the DNA construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire DNA construct, in accordance with standard techniques. The DNA construct may also be prepared by polymerase chain reaction using specific primers, for instance as described in U.S. Pat. No. 4,683,202 or R. K. Saiki et al., Science 239, 1988, pp. 487-491.

The invention further relates to a recombinant expression vector into which the DNA construct of the invention is inserted. This may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

In the vector, the DNA sequence encoding the endoglucanase should be operably connected to a suitable promoter and terminator sequence. The promoter may be any DNA sequence which shows transcriptional activity in the host cell of choice and may be derived from genes encoding proteins either homologous or heterologous to the host cell. The procedures used to ligate the DNA sequences coding for the endoglucanase, the promoter and the terminator, respectively, and to insert them into suitable vectors are well known to persons skilled in the art (cf., for instance, Sambrook et al., op.cit.).

The invention also relates to a host cell which is transformed with the DNA construct or the expression vector of the invention. The host cell may for instance belong to a species of Aspergillus, most preferably Aspergillus oryzae or Aspergillus niger. Fungal cells may be transformed by a process involving protoplast formation and transformation of the protoplasts followed by regeneration of the cell wall in a manner known per se. The use of Aspergillus as a host microorganism is described in EP 238,023 (of Novo Industri A/S), the contents of which are hereby incorporated by reference. The host cell may also be a yeast cell, e.g. a strain of Saccharomyces cerevisiae.

Alternatively, the host organism may be a bacterium, in particular strains of Streptomyces and Bacillus, and E. coli. The transformation of bacterial cells may be performed according to conventional methods, e.g. as described in Sambrook et al., Molecular Cloning: A Laboratory Manual, Cold Spring Harbor, 1989.

The present invention further relates to a process for producing an endoglucanase enzyme of the invention, the process comprising culturing a host cell as described above in a suitable culture medium under conditions permitting the expression of the endoglucanase enzyme, and recovering the endoglucanase enzyme from the culture. The medium used to culture the transformed host cells may be any conventional medium suitable for growing the host cells in question. The expressed endoglucanase may conveniently be secreted into the culture medium and may be recovered therefrom by well-known procedures including separating the cells from the medium by centrifugation or filtration, precipitating proteinaceous components of the medium by means of a salt such as ammonium sulphate, followed by chromatographic procedures such as ion exchange chromatography, affinity chromatography, or the like.

By employing recombinant DNA techniques as indicated above, techniques of protein purification, techniques of fermentation and mutation or other techniques which are well known in the art, it is possible to provide endoglucanases of a high purity.

The cellulase preparation or endoglucanase enzyme of the invention may conveniently be added to cellulose-containing fabrics together with other detergent materials during soaking, washing or rinsing operations. Accordingly, in another aspect, the invention relates to a detergent additive comprising the cellulase preparation or endoglucanase enzyme of the invention. The detergent additive may suitably be in the form of a non-dusting granulate, stabilized liquid or protected enzyme. Non-dusting granulates may be produced e.g. according to U.S. Pat. Nos. 4,106,991 and 4,661,452 (both to Novo Industri A/S) and may optionally be coated by methods known in the art. Liquid enzyme preparations may, for instance, be stabilized by adding a polyol such as propylene glycol, a sugar or sugar alcohol, lactic acid or boric acid according to established methods. Other enzyme stabilizers are well known in the art. Protected enzymes may be prepared according to the method disclosed in EP 238,216.

The detergent additive may suitably contain 1-500, preferably 5-250, most preferably 10-100 mg of enzyme protein per gram of the additive. It will be understood that the detergent additive may further include one or more other enzymes, such as a protease, lipase, peroxidase or amylase, conventionally included in detergent additives.

According to the invention, it has been found that when the protease is one which has a higher degree of specificity than Bacillus lentus serine protease, an increased storage stability of the endoglucanase enzyme is obtained. (For the present purpose, a protease with a higher degree of specificity than B. lentus serine protease is one which degrades human insulin to fewer components than does the B. lentus serine protease under the following conditions: 0.5 ml of a 1 mg/ml solution of human insulin in B and R buffer, pH 9.5, is incubated with 75 μl enzyme solution of 0.6 CPU [cf. Novo Nordisk Analysis Methods No. AF 228/1] per liter for 120 min. at 37° C., and the reaction is quenched with 50 microliters 1N HCl). Examples of such proteases are subtilisin Novo or a variant thereof (e.g. a variant described in U.S. Pat. No. 4,914,031), a protease derivable from Nocardia dassonvillei NRRL 18133 (described in WO 88/03947), a serine protease specific for glutamic and aspartic acid, producible by Bacillus licheniformis (this protease is described in detail in co-pending International patent application No. PCT/DK91/00067), or a trypsin-like protease producible by Fusarium sp. DSM 2672 (this protease is described in detail in WO 89/06270).

In a still further aspect, the invention relates to a detergent composition comprising the cellulase preparation or endoglucanase enzyme of the invention.

Detergent compositions of the invention additionally comprise surfactants which may be of the anionic, non-ionic, cationic, amphoteric, or zwitterionic type as well as mixtures of these surfactant classes. Typical examples of anionic surfactants are linear alkyl benzene sulfonates (LAS), alpha olefin sulfonates (AOS), alcohol ethoxy sulfates (AES) and alkali metal salts of natural fatty acids. It has, however, been observed that the endoglucanase is less stable in the presence of anionic detergents and that, on the other hand, it is more stable in the presence of non-ionic detergents or certain polymeric compounds such as polyvinylpyrrolidone, polyethylene glycol or polyvinyl alcohol. Consequently, the detergent composition may contain a low concentration of anionic detergent and/or a certain amount of non-ionic detergent or stabilizing polymer as indicated above.

Detergent compositions of the invention may contain other detergent ingredients known in the art as e.g. builders, bleaching agents, bleach activators, anti-corrosion agents, sequestering agents, anti soil-redeposition agents, perfumes, enzyme stabilizers, etc.

The detergent composition of the invention may be formulated in any convenient form, e.g. as a powder or liquid. The enzyme may be stabilized in a liquid detergent by inclusion of enzyme stabilizers as indicated above. Usually, the pH of a solution of the detergent composition of the invention will be 7-12 and in some instances 7.0-10.5. Other detergent enzymes such as proteases, lipases or amylases may be included the detergent compositions of the invention, either separately or in a combined additive as described above.

The softening, soil removal and color clarification effects obtainable by means of the cellulase preparation of the invention generally require a concentration of the cellulase preparation in the washing solution of 0.0001-100, preferably 0.0005-60, and most preferably 0.01-20 mg of enzyme protein per liter. The detergent composition of the invention is typically employed in concentrations of 0.5-20 g/l in the washing solution. In general, it is most convenient to add the detergent additive in amounts of 0.1-5% w/w or, preferably, in amounts of 0.2-2% of the detergent composition.

In a still further aspect, the present invention relates to a method of reducing the rate at which cellulose-containing fabrics become harsh or of reducing the harshness of cellulose-containing fabrics, the method comprising treating cellulose-containing fabrics with a cellulase preparation or endoglucanase enzyme as described above. The present invention further relates to a method providing color clarification of colored cellulose-containing fabrics, the method comprising treating colored cellulose-containing fabrics with a cellulase preparation or endoglucanase, and a method of providing a localized variation in color of colored cellulose-containing fabrics, the method comprising treating colored cellulose-containing fabrics with a cellulase preparation or endoglucanase of the invention. The methods of the invention may be carried out by treating cellulose-containing fabrics during washing. However, if desired, treatment of the fabrics may also be carried out during soaking or rinsing or simply by adding the cellulase preparation or the endoglucanase enzyme to water in which the fabrics are or will be immersed.

According to the invention, it has been found that the drainage properties of paper pulp may be significantly improved by treatment with the endoglucanase of the invention without any significant concurrent loss of strength. Consequently, the present invention further relates to a method of improving the drainage properties of pulp, the method comprising treating paper pulp with a cellulase preparation or an endoglucanase enzyme according to the invention. Examples of pulps which may be treated by this method are waste paper pulp, recycled cardboard pulp, kraft pulp, sulphite pulp, or thermomechanical pulp and other high-yield pulps.

The present invention is described in further detail with reference to currently preferred embodiments in the following examples which are not intended to limit the scope of the invention in any way.

### EXAMPLES

### Example 1

Isolation of a ˜43 kD Endoglucanase from Humicola insolens

1. Preparation of a Rabbit Antibody Reactive with a ˜43 kD Endoglucanase Purified from Humicola insolens Cellulase Mixture

Cellulase was produced by cultivating Humicola insolens DSM 1800, as described in U.S. Pat. No. 4,435,307, Example 6. The crude cellulase was recovered from the culture broth by filtration on diatomaceous earth, ultrafiltration and freeze-drying of the retentate, cf. Examples 1 and 6 of U.S. Pat. No. 4,435,307.

The crude cellulase was purified as described in WO 89/09259, resulting in the fraction F1P1C2 which was used for the immunization of mice. The immunization was carried out 5 times at bi-weekly intervals, each time using 25 micrograms protein including Freund's Adjuvant.

Hybridoma cell lines were established as described in Ed Harlow and David Lane, Antibodies. A Laboratory Manual, Cold Spring Harbor Laboratory 1988. The procedure may briefly be described as follows:

After bleeding the mouse and showing that the mouse serum reacts with proteins present in the F1P1C2 fraction, the spleen was removed and homogenized and then mixed with PEG and Fox-river myeloma cells from Hyclone, Utah, USA.

The hybridomas were selected according to the established HAT screening procedure.

The recloned hybridoma cell lines were stabilized. The antibodies produced by these cell lines were screened and selected for belonging to the IgG1 subclass using a commercial mouse monoclonal typing kit from Serotec, Oxford, England. Positive antibodies were then screened for reactivity with F1P1C2 in a conventional ELISA, resulting in the selection of F4, F15 and F41 as they were all very good in ELISA response but were found to have different response in immunoblotting using crude H. insolens, DSM 1800, cellulase in SDS-PAGE followed by Western Blot, indicating that they recognized different epitopes.

The three antibodies were produced in large quantities in the ascites fluid of CRBF₁ mice. The mouse gammaglobulin was purified from ascites fluid by protein A purification using protein A coupled to Sepharose (Kem.En.Tek., Copenhagen, Denmark).

The different monoclonal gammaglobulins were tested for response in a sandwich ELISA using each monoclonal antibody as the catching antibody, various HPLC fractions of Celluzyme as the antigen, and a rabbit antibody raised against endoglucanase B from Celluzyme as the detection antibody.

To visualize binding in the ELISA, a porcine antibody against rabbit IgG covalently coupled to peroxidase from Dakopatts (Copenhagen, Denmark) and was visualized with OPD (1,2-phenylenediamine, dihydrochoride)/H₂O₂.

The highest ELISA response was obtained with the monoclonal antibody F41 which was therefore used in the immunoaffinity purification steps.

The purified mouse gammaglobulin F41 was coupled to 43 g of CNBr-activated Sepharose 4B as described by the manufacturer (Pharmacia, Sweden) followed by washing.

2. Immunoaffinity Purification of ˜43 kD Endoglucanase from a H. insolens Cellulase Mixture

H. insolens cellulase mixture (as described above) was diluted to 3% dry matter, and the pH was adjusted to 3.5 in 15 min. at 4° C. The precipitate was removed by filtration after adjusting the pH to 7.5. Then sodium sulphate was added to precipitate the active enzyme and this was done at 40° C. (260 grams per kg at pH 5.5). The precipitate was solubilized with water and filtered. The acid treatment was repeated. Finally, the product was filtered and concentrated by ultrafiltration using a polyvinylsulphonate membrane with a 10,000 Mw cut-off.

The cellulase product was then diluted to 3% dry matter, adjusting the pH to 9.0, and subjected to anion exchange chromatography on a DEAE-Sepharose column as recommended by the manufacturer (Pharmacia, Sweden).

The protease-free cellulase product was applied on the F 41 gammaglobulin-coupled Sepharose column described above at pH 8.0 in sodium phoshate buffer.

After application the column was washed with the same buffer containing 0.5 M sodium chloride. The column was then washed with 0.1 M sodium acetate buffer containing 0.5 M sodium chloride, pH 4.5, after which the column was washed in 5 mM sodium acetate buffer, pH 4.5. Finally, the −43 kD endoglucanase was eluted with 0.1 M citric acid.

Total yield: 25 mg with an endoglucanase activity of 1563 CMC-endoase units.

The eluted protein migrates as a single band in SDS-PAGE with an apparent MW of ˜43 kD and a pI after isoelectric focusing of about 5.0 to 5.2.

Inactive protein was removed by reverse phase purification.

Inactive and active protein was separated by HPLC using a gradient of 2-propanol. Inactive protein elutes at about 25% 2-propanol and the active ˜43 kD endoglucanase elutes at 30% 2-propanol, the active endoglucanase being detectable by a CMC-Congo Red clearing zone.

In this way, a total of 0.78 mg active protein was recovered with 122 CMC endoase units. This procedure was repeated 30 times.

The ˜43 kD endoglucanase was recovered by first freeze-drying to remove the TFA and propanol and then solubilizing in phosphate buffer.

The endoglucanase activity of the purified material was 156 CMC-endoase units per mg protein and the total yield including freze-drying was 65% of the endoglucanase activity.

The thus obtained ˜43 kD enzyme was used to immunize rabbits according to the procedure described by N. Axelsen et al. in A Manual of Quantitative Immunolelectrophoresis, Blackwell Scientific Publications, 1973, Chapter 23. Purified immunoglobulins were recovered from the antisera by ammonium sulphate precipitation followed by dialysis and ion exchange chromatography on DEAE-Sephadex in a manner known per se. Binding of purified immunoglobulin to the endoglucanase was determined, and the rabbit immunoglobulin AS 169 was selected for further studies.

2. Characterization of the −43 kD Endoglucanase:

Amino acid composition: Using total hydrolysis, the following composition was obtained after amino acid analysis:

| | |
|---|---|
| Asp | 17 |
| Asn | 15 |
| Thr | 25 |
| Ser | 29 |
| Glu | 6 |
| Gln | 13 |
| Pro | 21 |
| Gly | 32 |
| Ala | 23 |
| Cys | 20 |
| Val | 14 |
| Met | 1 |
| Ile | 7 |
| Leu | 8 |
| Tyr | 6 |
| Phe | 15 |
| Lys | 9 |
| His | 2 |
| Trp | 9 |
| Arg | 12 |

The Mw of the non-glycosylated protein was estimated to be 30,069 based on the amino acid composition. The glycosylation was measured to

| | |
|---|---|
| Galactose | 10 |
| Mannose | 28 |

corresponding to a Mw of 6,840, resulting in a total Mw of the endoglucanase of 36,900 (+/−2,400). The extinction coefficient per mole was estimated as follows:

| | |
|---|---|
| Tryptophan | 9 times 5690 |
| Tyrosine | 6 times 1280 |
| Cysteins | 20 times 120 |
| total | 61290 per mole. |

Extinction coefficients are 1.66 at 280 nm corresponding to 1 mg protein per ml. (Reference: S. C. Gill and P. Hippel, Anal. Biochemistry 182, 312-326 (1989))

The amino acid sequence was determined on an Applied Biosystems 475A Protein Sequenator using Edman degradation. Only one sequence indicated the purity of the protein. The amino acid sequence is shown in SEQ ID NO: 2.

Enzyme Properties:

The enzyme is stable between pH 3 and 9.5.

The enzyme does not degrade highly crystalline cellulose or the substrate cellobiose beta-p-nitrophenyl, (Cellobiohydrolase substrate), but degrades amorphous cellulose mainly to cellobiose, cellotriose and cellotetraose, indicating that the enzyme may be used to produce cellodextrins from insoluble amorphous cellulose.

The enzyme is active between pH 6.0 and 10.0 with a maximum activity at about 50° C.

### Example 2

Cloning and Expression of the ˜43 kD Endoglucanase in Aspergillus oryzae

Partial cDNA:

A cDNA library was made from Humicola insolens strain DSM 1800 mRNA (Kaplan et al. (1979) Biochem. J. 183, 181-184) according to the method of Okayama and Berg (1982) Mol. Cell. Biol. 2, 161-170. This library was screened by hybridization with radioactively labelled oligonucleotides to filters with immobilized DNA from the recombinants (Gergen et al. (1979) Nucleic Acids Res. 7, 2115-2136). The oligonucleotide probes were made on the basis of amino acid sequences of tryptic fragments of the purified ˜43 kD endoglucanase. A colony was found to hybridize to three different probes (NOR 1251, 2048, and 2050) and was isolated. The sequence showed that the inserted 680 bp cDNA coded for the C-terminal 181 amino acids of the −43 kD protein and the 31 nontranslated mRNA. A 237 bp long Pvu I-Xho I fragment from this clone was used to probe a Northern blot (as described in Sambrook et al, op. cit., p. 7.40-7.42 and p. 7.46-7.48.) with H. insolens mRNA and it was shown that the entire ˜43 kD mRNA has a length of app. 1100 bp. The same 237 bp fragment was used to probe a genomic library from the same strain.

Genomic Clone:

A Humicola insolens strain DSM 1800 genomic library was made from total DNA prepared by the method of Yelton (M. M. Yelton et al. (1984) Proc. Natl. Acad. Sci. USA. 81. 1470-1474) and partially digested with Sau 3A. Fragments larger than 4 kb were isolated from an agarose gel and ligated to pBR 322 digested with Bam HI and dephosphorylated. The ligation products was transformed into E. coli MC1000 (Casadaban and Cohen (1980). J. Mol. Biol., 138, 179-207) made r⁻m⁺ by conventional methods. 40.000 recombinants were screened with the 237 bp Pvu I-Xho I partial cDNA fragment described in the paragraph “partial cDNA”. 2 colonies that contained the entire ˜43 kD endoglucanase sequence were selected and the gene was sequenced by the dideoxy method using the Sequenase® kit (United States Biochemical Corporation) according to the manufacturer's instructions. The sequence was identical to the sequence of the full length cDNA gene (see the paragraph “full length cDNA” below) except for one intron in the genomic gene.

The genomic gene was amplified by the PCR method using a Perkin-Elmer/Cetus DNA Amplification System according to the manufacturer's instructions. In the 5′ end of the gene the primer NOR 2378 was used. This primer is a 25 mer matching the 5′ untranslated end of the gene except for one C to T replacement generating a Bcl I site. In the 3′ end of the gene the primer NOR 2389 was used. This primer is a 26 mer of which 21 bases match the 3′ untranslated part of the gene and the 5 bases in the 5′ end of the primer completes a Sal I site.

The Aspergillus expression vector pToC 68 was constructed from plasmid p775 (the construction of which is described in EP 238 023) by insertion of the following linkers

(SEQ ID NO:5)

| | |
|---|---|
| KFN 514: | 5′-AGCTGCGGCCGCAGGCCGCGGAGGCCA-3′ |

(SEQ ID NO:6)

| | |
|---|---|
| KFN 515: | 3′-CGCCGGCGTCCGGCGCCTCCGGTTCGA-5′ |
| | SacII HindIII |

(SEQ ID NO:7)

| | |
|---|---|
| | EcoRI NotI StiI |
| KFN 516: | 5′-AATTCGCGGCCGCGGCCATGGAGGCC-3′ |

(SEQ ID NO:8)

| | |
|---|---|
| KFN 519: | 3′-GCGCCGGCGCCGGTACCTCCGGTTAA-5′ |
| | NcoI |

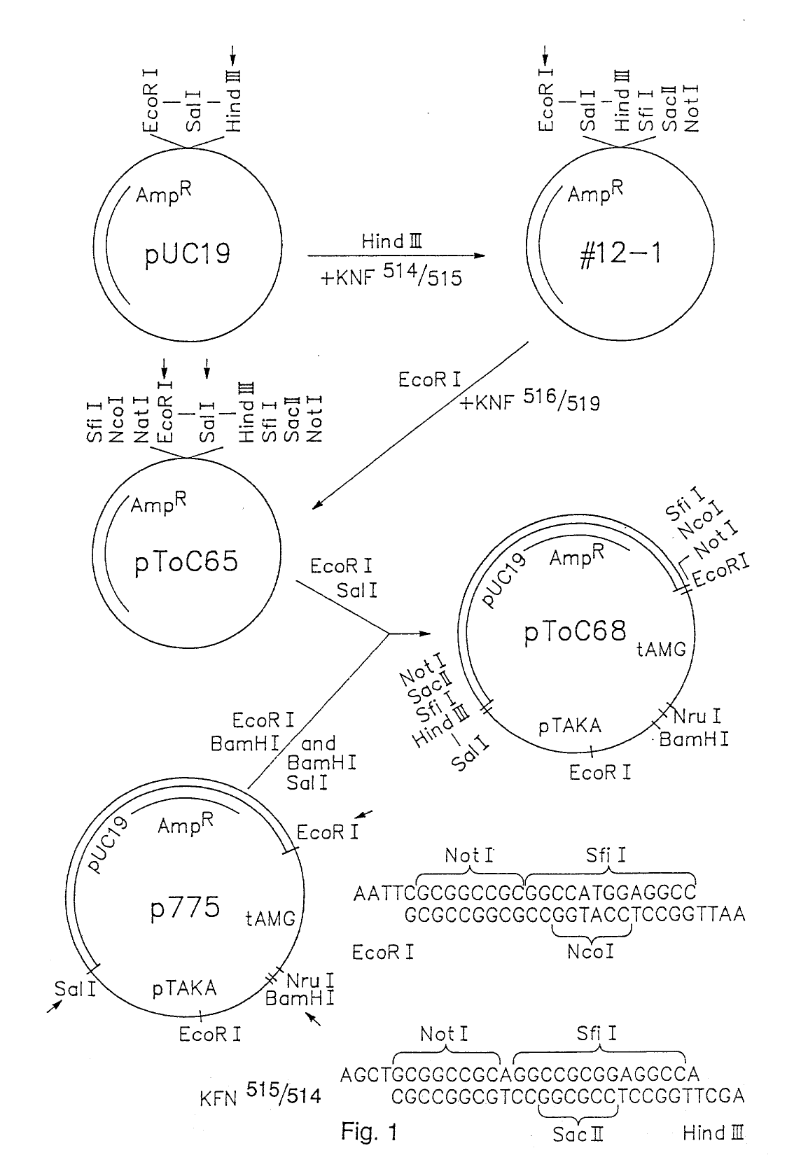

The construction of pToC is shown in FIG. 1.

The PCR fragment obtained above was digested with Bcl I and Sal I and inserted into pToC 68 digested with Bam HI and Xho I. The insert of the resulting plasmid (pCaHj 109) was sequenced and shown to be identical to the original clone.

Full Length cDNA:

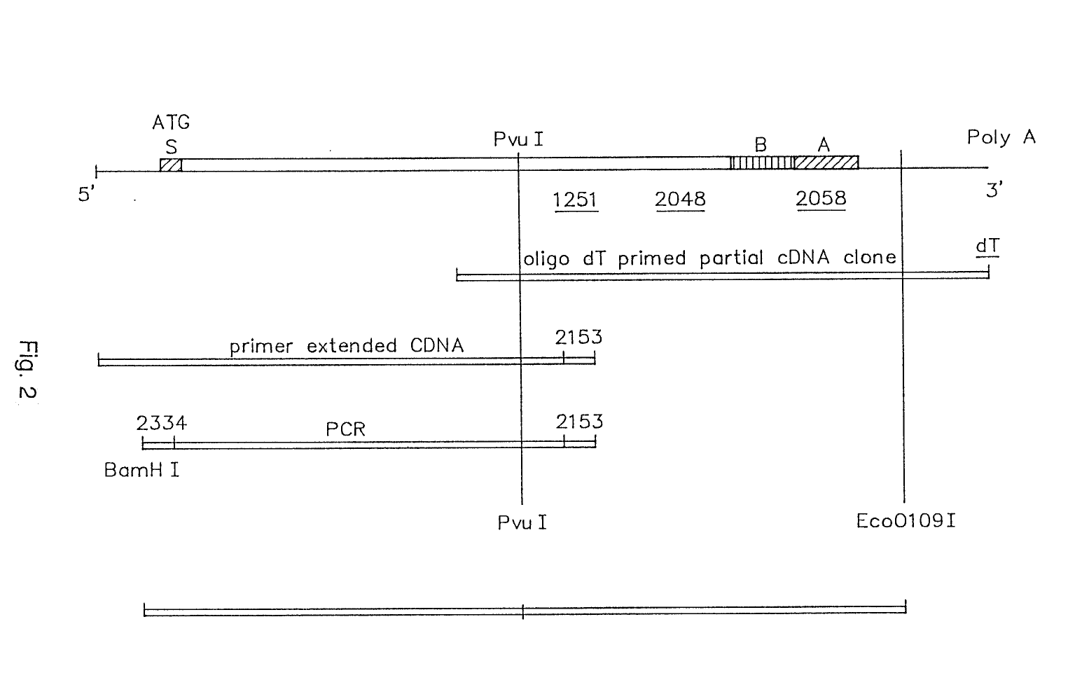

First strand cDNA was synthesized from a specific primer within the known sequence (NOR 2153), and second strand synthesis was made by the method described by Gubler and Hoffman (1983) GENE 25, 263-269. The sequence of the genomic gene made it possible to design a PCR primer to catch the 5′ part of the mRNA and at the same time introduce a Bam HI site right in front of the ATG start codon (NOR 2334). By using this primer at the 5′ end and NOR 2153 again at the 3′ end PCR was performed on the double stranded cDNA product. The full length coding part of the PCR-cDNA was then constructed by cloning the 5′ Bam HI-Pvu I fragment from the PCR reaction together with the 3′ Pvu I-Eco 0109, filled out with Klenow polymerase to make it blunt ended, into Bam HI-Nru I cut Aspergillus expression vector pToC 68 (FIG. 1), and the sequence of the inserted DNA was checked (pSX 320) (cf. FIG. 2). The sequence of the full length cDNA is shown in SEQ ID NO: 1.

Oligonucleotide primers used:

| | |
|---|---|
| NOR 1251: | |
| 5′-AAYGCYGACAAAYCC-3′ | (SEQ ID NO:9) |
| NOR 2048: | |
| 5′-AACGAYGAYGGNAAYTTCCC-3′ | (SEQ ID NO:10) |
| NOR 2050: | |
| 5′-AAYGAYTGGTACCAYCARTG-3′ | (SEQ ID NO:11) |
| NOR 2153: | |
| 5′-GCGCCAGTAGCAGCCGGGCTTGAGGG-3′ | (SEQ ID NO:12) |
| NOR 2334: | |
| 5′-ACGTCTCAACTCGGATCCAAGATGCGTT-3′ | (SEQ ID NO:13) |
| Bam HI | |
| NOR 2378: | |
| 5′-CTCAACTCTGATCAAGATGCGTTCC-3′ | (SEQ ID NO:14) |
| Bcl I | |
| NOR 2389: | |
| 5′-TGTCGACCAGTAAGGCCCTCAAGCTG-3′ | (SEQ ID NO:15) |
| Sal I | |

| Nomenclature: | |
|---|---|
| Y: | Pyrimidine (C + T) |
| R: | Purine (A + G) |
| N: | All four bases |

Enhanced: Changes or insertions relative to original sequence.

Underlined: Restriction site introduced by PCR.

Expression of the −43 kD endoglucanase:

The plasmid pSX 320 was transformed into Aspergillus oryzae A1560-T40, a protease deficient derivative of A. oryzae IFO 4177, using selection on acetamide by cotransformation with pToC 90 harboring the amdS gene from A. nidulans as a 2.7 kb Xba I fragment (Corrick et al. (1987), GENE 53, 63-71) on a pUC 19 vector (Yannisch-Perron et al. (1985), GENE 33, 103-119). Transformation was performed as described in the published EP patent application No. 238 023. A number of transformants were screened for co-expression of ˜43 kD endoglucanase. Transformants were evaluated by SDS-PAGE (p.3) and CMC endoglucanase activity.

The plasmid containing the genomic gene (pCaHj 109) was transformed into Aspergillus oryzae A1560-T40 by the same procedure. Evaluation of the transformants showed that the level of expression was similar to that of the cDNA transformants.

The purified ˜43 kD endoglucanase was analyzed for its N-terminal sequence and carbohydrate content. The N-terminal amino acid sequence was shown to be identical to that of the HPLC purified ˜43 kD endoglucanase. The carbohydrate content differs from that of the HPLC purified ˜43 kD enzyme in that the recombinant enzyme contains 10+/−8 galactose sugars per mol rather than glucose.

### Example 3

Isolation of Fusarium oxysporum Genomic DNA

A freeze-dried culture of Fusarium oxysporum was reconstituted with phosphate buffer, spotted 5 times on each of 5 FOX medium plates (6% yeast extract, 1.5% K₂HPO₄, 0.75% MgSO₄ 7H₂O, 22.5% glucose, 1.5% agar, pH 5.6) and incubated at 37° C. After 6 days of incubation the colonies were scraped from the plates into 15 ml of 0.001% Tween-80 which resulted in a thick and cloudy suspension.

Four 1-liter flasks, each containing 300 ml of liquid FOX medium, were inoculated with 2 ml of the spore suspension and were incubated at 30° C. and 240 rpm. On the 4th day of incubation, the cultures were filtered through 4 layers of sterile gauze and washed with sterile water. The mycelia were dried on Whatman filter paper, frozen in liquid nitrogen, ground into a fine powder in a cold morter and added to 75 ml of fresh lysis buffer (10 mM Tris-Cl 7.4, 1% SDS, 50 mM EDTA, 100 microliters DEPC). The thoroughly mixed suspension was incubated in a 65° C. waterbath for 1 hour and then spun for 10 minutes at 4000 RPM and 5° C. in a bench-top centrifuge. The supernatant was decanted and EtOH precipitated. After 1 hour on ice the solution was spun at 19,000 rpm for 20 minutes. The supernatant was decanted and isopropanol precipitated. Following centrifugation at 10,000 rpm for 10 minutes, the supernatant was decanted and the pellets allowed to dry.

One milliliter of TER solution (10 mM Tris-HCl, pH 7.4, 1 mM EDTA 2000 100 micrograms RNAseA) was added to each tube, and the tubes were stored at 4° C. for two days. The tubes were pooled and placed in a 65° C. waterbath for 30 minutes to suspend non-dissolved DNA. The solution was extracted twice with phenol/CHCl₃/isoamyl alcohol, twice with CHCl₃/isoamyl alcohol and then ethanol precipitated. The pellet was allowed to settle and the EtOH was removed. 70% EtOH was added and the DNA was stored overnight at −20° C. After decanting and drying, 1 ml of TER was added and the DNA was dissolved by incubating the tubes at 65° C. for 1 hour. The preparation yielded 1.5 mg of genomic DNA.

Cloning of Fusarium oxysporum ˜43 kD Endoglucanase

To isolate the Fusarium homologue to the Humicola ˜43 kD endoglucanase, a fragment was first obtained by PCR (as described IN U.S. Pat. No. 4,683,195 and U.S. Pat. No. 4,683,202) and cloned. This product was then sequenced and primers to be used as library probes and for PCR amplification were constructed. These oligonucleotides were used to isolate the corresponding clone from a cDNA library.

PCR was used to isolate partial length cDNA and genomic fragments of the 43 kD homologue. Seven different combinations of highly degenerate oligonucleotides (see table below) were used in PCR reactions with either cDNA or genomic DNA as templates. Only one combination yielded partial clones of the Fusarium 43 kd homologue. Two separate sets of PCR conditions were used for each oligonucleotide pair; the first set was designed to make very little product but with very high specificity. Various factors ensured specificity in this set of 28 cycles: The annealing temperature of 65° C. was very high for these oligonucleotides; the time at annealing temperature was set for only 30 seconds; 20 picomoles of each degenerate primer mixture was used per 100 microliter reaction. The oligonucleotides used contained only the degenerate region without a “cloning element”; 1 unit of Amplitaq™ polymerase (Perkin-Elmer Cetus) was used per 100 microliter reaction; and EDTA was added to reaction tubes at the end of the final 10 minute 72° C. incubation to prevent extension from mismatched primers at cooler temperatures following the PCR cycles. Products of the first set of cycles would not be expected to be visible by ethidium bromide staining in agarose gel electrophoresis due to the low efficiency of amplification required to ensure high specificity. The second set of amplifications was, however, designed to efficiently amplify products from the first set. Factors ensuring this include: lowering the annealing temperature to 55° C.; lengthening the time of annealing to 1 minute; increasing the amount of oligonucleotides to 100 picomoles of each mixture per 100 microliter reaction; utilizing a different set of oligonucleotides which include a “Prime” cloning element along with the degenerate portion (increasing the melting temperature dramatically) and by using 2.5 units of Amplitaq polymerase per 100 microliter reaction.

PCR reactions were set up as recommended by Perkin-Elmer Cetus. A master mix was made for each of 2 DNA sources, genomic and cDNA. This was comprised of 1× PCR buffer (10 mM Tris/HCl pH 8.3, 50 mM KCl, 1.5 mM MgCl₂, 0.01% gelatin, Perkin-Elmer Cetus), 0.2 mM deoxynucleotides (Ultrapure™ dNTP 100 mM solution, Pharmacia), 1 unit Amplitaq™ polymerase (Perkin Elmer Cetus) and 0.5 microgram genomic DNA or 50 ng cDNA per 100 microliter reaction mixture volume, and deionized water to bring volume up to 98 microliters per 100 μl reaction. To labeled 0.5 tubes (Eppendorf) were added 20 picomoles (1 microliter of a 20 picomole/microliter concentration) of each oligonucleotide mixture (see table below). These were placed in a Perkin-Elmer Cetus thermocycler at 75° C. along with the master mixes and light mineral oil also in 0.5 ml tubes. Ninety eight microliters of the appropriate master mix and 55 microliters light mineral oil were added to each tube with oligonucleotides. The reactions were then started in a step-cycle file (see chart below for parameters). At the end of the final 72° C. incubation, 50 microliters of a 10 mM EDTA pH 8.0 solution was added to each tube and incubated for a further 5 minutes at 72° C.

Table of oligonucleotide pairs used in 43 kD homologue PCR:

| | | | | expected |
|---|---|---|---|---|
| | | | oligos for | size in |
| reaction | | | second set | degenerate |
| | ge- | oligos for | degenerate only | with base |
| cDNA | nomic | first set | “prime” | pairs |
| 1 | 11 | ZC3485 vs ZC3558 | ZC3486 vs ZC3559 | 288 |
| 2 | 12 | ZC3485 vs ZC3560 | ZC3486 vs ZC3561 | 510 |
| 3 | 13 | ZC3485 vs ZC3264 | ZC3486 vs ZC3254 | 756 |
| 4 | 14 | ZC3556 vs ZC3560 | ZC3557 vs ZC3561 | 159 |
| 5 | 15 | ZC3556 vs ZC3264 | ZC3557 vs ZC3254 | 405 |
| 6 | 16 | ZC3556 vs ZC3465 | ZC3557 vs ZC3466 | 405 |
| 7 | 17 | ZC3485 vs ZC3465 | ZC3486 vs ZC3466 | 756 |

Note:
See oligonucleotide table for oligonucleotide sequences
Conditions for PCR step-cycle file were:
SET 1:
94° C. 1 min
28 × 65° C. 30 sec
72° C. 2 min
72° C. 10 min
SET 2:
94° C. 1 min
28 × 55° C. 1 min
72° C. 2 min
72° C. 10 min

Following the first set of PCR cycles, DNA was purified from the reaction mixtures by isopropyl alcohol precipitation for use in the second set of cycles. Most of the light mineral oil was removed from the top of each sample before transferring the sample to a new labeled tube. Each tube was then extracted with an equal volume PCI (49% phenol: 49% chloroform: 2% isoamyl alcohol) and then with an equal volume of chloroform. DNA was then precipitated from the reactions by adding: 75 microliters 7.5 M ammonium acetate, 1 microliter glycogen and 226 microliters isopropyl alcohol. Pellets were resuspended in 20 microliters deionized water. Two microliters of each resuspension were placed into labeled tubes for the second round of PCR amplifications along with 100 picomoles (5 microliters of a 20 picomole/microliter concentration) of each new primer mixture (see table above). A master mix was made as described above except for excluding Alegenomic and cDNA templates and compensating for increased oligonucleotide and DNA volumes in the reaction tubes by decreasing the volume of water added. Reactions and cycles were set up as described above (see table above).

After the 28 cycles were completed, light mineral oil was removed from the tops of the samples, and the PCR mixtures were removed to new tubes. Ten microliters of each sample were spotted onto parafilm and incubated at 45° C. for approximately 5 minutes to allow the sample to decrease in volume and to allow the parafilm to absorb any residual light mineral oil. The drops were then combined with 2 microliters 6× loading dye and electrophoresed on 1% agarose (Seakem GTG™, FMC, Rockland, Me.) gel. A single band of approximately 550 base pairs was found in reaction number 2 where the template was cDNA. A band of approximately 620 base pairs in reaction number 12 where the template was genomic DNA. These reactions were primed with oligonucleotides ZC3486 and ZC3561 (Table 1). This was very close to the 510 base pair PCR product predicted from comparison with the Humicola 43 kD sequence. The synthesis of a larger product in the reaction with genomic template is due to the presence of an intron within this region. The agarose containing these 2 bands was excised and DNA was extracted utilizing a Prep-A-Gene™ kit (BioRad) following manufacturers instructions. DNA was eluted with 50 microliters deionized water and precipitated with 5 μl 3M sodium acetate, 1 microliter glycogen and 140 microliters ethanol. The DNA pellet was dried and resuspended in a volume of 7 microliters TE (10 mM Tris-HCL pH 8.0, 1 mM EDTA).

The PCR fragments were cloned into pBS sk-′vector was constructed by first digesting pBluescript II sk-(Stratagene, La Jolla, Calif.) with Eco RI and gel purifying cut plasmid from 0.8% seaplaque GTG™ agarose (FMC) with a Pre-A-Gene™ kit (BioRad) following the manufacturer's instructions. Oligonucleotides ZC1773 and ZC1774 (Table 1) were annealed by mixing 2 picomoles of each oligonucleotide, bringing up the reaction volume to 4 microliters with deionized water then adding 0.5 microliter annealing buffer (200 mM Tris-HCl pH 7.6, 50 mM MgCl₂) and bringing the temperature up to 65° C. for 30 seconds and slowly cooling to 20° C. in 20 minutes in a Perkin-Elmer Cetus PCR thermocycler. The oligonucleotides were then ligated into the Eco RI digested pBluescript vector by mixing: 5.5 microliters deionized water, 2 microliters anealed oligonucleotides, 1 microliter of a 1:3 dilution in deionized water of digested vector, 1 microliter 10× T4 DNA ligase buffer (Boehringer-Mannheim Biochemicals, Indianapolis Ind.) and 0.5 T4 DNA ligase (Gibco-BRL), and incubating the mixture at 16° C. for 2.5 hours. The ligation mixture was then brought up to a volume of 100 microliters with deionized water and extracted with PCI and chloroform. To increase electroporation efficiency, DNA was then precipitated with 50 microliters ammonium acetate, 1 microliter glycogen and 151 microliters isopropanol. One microliter of a 10 microliter resuspension in deionized water was electroporated into E. coli DH10-B electromax cells (Gibco-BRL) using manufacturer's instructions, in a Bio-Rad electroporation apparatus. Immediately following the electroporation, 1 ml of 2XYT (per liter: 16 g tryptone, 10 g yeast extract, 10 g NaCl) broth was added to the cuvet and mixed. Various dilutions were plated onto 100 mm LB plates (per liter: 10 g tryptone, 8 g yeast extract, 5 g NaCl, 14.5 g agar) with 100 micrograms/ml ampicillin, and coated with 100 microliters of 20 mg/ml X-Gal (5-Bromo-4 Chloro-3-Indolyl-b-D-galactropyranoside; Sigma, St. Louis, Mo.) in dimethylformamide and 20 microliters of 1 M IPTG (Sigma). After overnight growth various blue and white colonies were analyzed by PCR for small inserts using the oligonucleotides ZC3424 (bluescript reverse primer) and ZC3425 (T7 promoter primer) (Table 1), following conditions outlined above for screening bacterial plugs. After an initial 1 minute 45 seconds at 94° C. denaturation, 30 cycles of 94° C. for 45 seconds, 40° C. for 30 seconds and 72° C. for 1 minute were performed. Upon agarose gel electrophoresis of the PCR products, 1 blue colony giving a PCR band consistent with a small insert in the pBluescript cloning region was chosen for DNA purification and was grown up overnight in a 100 ml liquid culture in TB (per liter: 12 g tryptone, 24 g yeast extract, 4 ml glycerol, autoclave. Then add 100 ml of 0.17 M KH₂PO₄, 0.72 M K₂HPO₄; Sambrook et al., Molecular Cloning, 2nd Ed., 1989, A.2) with 150 micrograms/ml ampicillin. DNA was isolated by alkaline lysis and PEG precipitation (Sambrook et al., Molecular Cloning 2nd ed., 1.38-1.41, 1989). Sequence analysis showed the correct oligonucleotide to be inserted while maintaining the beta-galactosidase gene present in pBluescript vectors in frame with the promoter. Fifty micrograms of the DNA preparation was digested with Eco RI, PCI and chloroform extracted, and precipitated with sodium acetate and ethanol. The DNA pellet was resuspended in 50 microliters deionized water. Digested pBS sk-1 was cut back with T4 DNA polymerase (Gibco-BRL) by adding 40 microliters 10× T4 DNA polymerase buffer (0.33 M Tri/acetate pH 8.0, 0.66 M potassium acetate, 0.1 M magnesium acetate, 5 mM dithiotheretiol, 5 mM BSA (New England Biolabs) 260 microliters deionized water, 40 microliters 1 mM dTTP (Ultrapure™, Pharmacia) and 40 microliters T4 DNA polymerase (1 U/μl) (Gibco-BRL) to 20 microliters of 1 mg/ml vector DNA. The mixture was incubated at 12° C. for 15 minutes, then at 75° C. for 10 minutes. To prepare the DNA for use in ligation, it was PCI and chloroform extracted and precipitated with sodium acetate and ethanol. The pellet was resuspended in 200 microliters deionized water, producing a concentration of 0.1 microgram/microliter.

To prepare the 43 kd homologue PCR products for insertion into the cut-back pBS sk-′ vector, they were cut back with T4 DNA polymerase (Gibco-BRL) in reaction volumes of 10 microliters with the inclusion of dATP instead of dTTP. The resulting DNA solutions were PCI and chloroform extracted and precipitated with sodium acetate, glycogen and ethanol. The DNA pellets were resuspended in 15 microliters deionized water. DNA samples of 7.5 microliters were ligated into 0.1 microgram cut back pBS sk-′ (0.1 μg/microliter) with 1 microliter 10× ligase buffer (Boehringer-Mannheim) and 0.5 microliter of T4 DNA ligase (Boehringer-Mannheim). The ligation mixtures were then brought up to a volume of 150 microliters with deionized water and extracted with PCI and chloroform. To increase electroporation efficiency, DNA was then precipitated with 15 microliters sodium acetate, 1 microliter glycogen and 166 microliters isopropanol. One microliter of a 10 microliter resuspension in deionized water was electroporated into E. coli DH10-B electromax cells (BRL) using a Bio-Rad electroporation apparatus, according to manufacturer's instructions. Immediately following the electroporation, 1 ml of SOB broth (per liter: 20 g tryptone, 5 g yeast extract, 10 ml 1 M NaCl, 2.5 ml 1 M KCl. Autoclave then add 10 ml 1 M MgCl₂ and 10 ml 1 M MgSO₄) was added to the cuvet, and the cell mixture was transferred to a 100 mm tube and incubated at 37° C. for 1 hour with airation. Various dilutions were plated onto 100 mm LB plates containing 100 μg/ml ampicillin and coated with 100 microliters of 20 mg/ml X-Gal (Sigma) in dimethylformamide and 20 microliters of 1 M IPTG (Sigma). Three white colonies of each of the 2 transformations, cDNA and genomic, were picked for sequencing. Sequence analysis showed the inserts to be highly homologous to the Humicola 43 kD cellulase. The genomic insert was identical to the cDNA except for the presence of an intron. Two 42-mer oligonucleotides ZC3709 and ZC3710 (Table 1) were designed from the sequence for use as library probes and PCR primers. The oligonucleotides were from opposite ends of the PCR product and were designed to hybridize opposite strands of the DNA so that they could be used as primers in a PCR reaction to test potential clones in the library screening.

Construction of a Fusarium oxysporum cDNA Library

Fusarium oxysporum was grown by fermentation and samples were withdrawn at various times for RNA extraction and cellulase activity analysis. The activity analysis included an assay for total cellulase activity as well as one for color clarification. Fusarium oxysporum samples demonstrating maximal color clarification were extracted for total RNA from which poly(A)+ RNA was isolated.

To construct a Fusarium oxysporum cDNA library, first-strand cDNA was synthesized in two reactions, one with and the other without radiolabelled dATP. A 2.5× reaction mixture was prepared at room temperature by mixing the following reagents in the following order: 10 microliters of 5× reverse transcriptase buffer (Gibco-BRL, Gaithersburg, Md.) 2.5 microliters 200 mM dithiothreitol (made fresh or from a stock solution stored at −70° C.), and 2.5 microliters of a mixture containing 10 mM of each deoxynucleotide triphosphate, (dATP, dGTP, dTTP and 5-methyl dCTP, obtained from Pharmacia LKB Biotechnology, Alameda, Calif.). The reaction mixture was divided into each of two tubes of 7.5 microliters. 1.3 microliters of 10 micro-Ci/microliter ³²P alpha-dATP (Amersham, Arlington Heights, Ill.) was added to one tube and 1.3 microliters of water to the other. Seven microliters of each mixture was transferred to final reaction tubes. In a separate tube, 5 micrograms of Fusarium oxysporum poly (A)⁺ RNA in 14 microliters of 5 mM Tris-HCl pH 7.4, 50 μM EDTA was mixed with 2 microliters of 1 microgram/microliter first strand primer (ZC2938 GACAGAGCACAGAATTCACTAGTGAGCTCT₁₅ (SEQ ID NO: 16)). The RNA-primer mixture was heated at 65° C. for 4 minutes, chilled in ice water, and centrifuged briefly in a microfuge. Eight microliters of the RNA-primer mixture was added to the final reaction tubes. Five microliters of 200 U/microliter Superscript™ reverse transcriptase (Gibco-BRL) was added to each tube. After gentle agitation, the tubes were incubated at 45° C. for 30 minutes. Eighty microliters of 10 mM Tris-HCl pH 7.4, 1 mM EDTA was added to each tube, the samples were vortexed, and briefly centrifuged. Three microliters was removed from each tube to determine counts incorporated by TCA precipitation and the total counts in the reaction. A 2 microliter sample from each tube was analyzed by gel electrophoresis. The remainder of each sample was ethanol precipitated in the presence of oyster glycogen. The nucleic acids were pelleted by centrifugation, and the pellets were washed with 80% ethanol. Following the ethanol wash, the samples were air dried for 10 minutes. The first strand synthesis yielded 1.6 μg of Fusarium oxysporum cDNA, a 33% conversion of poly(A)⁺ RNA into DNA.

Second strand cDNA synthesis was performed on the RNA-DNA hybrid from the first strand reactions under conditions which encouraged first strand priming of second strand synthesis resulting in hairpin DNA. The first strand products from each of the two first strand reactions were resuspended in 71 microliters of water. The following reagents were added, at room temperature, to the reaction tubes: 20 microliters of 5×second strand buffer (100 mM Tris pH 7.4, 450 mM KCl, 23 mM MgCl₂, and 50 mM (NH₄)₂(SO₄), 3 microliters of 5 mM beta-NAD, and microliter of a deoxynucleotide triphosphate mixture with each at 10 mM. One microliter of alpha-³²P dATP was added to the reaction mixture which received unlabeled dATP for the first strand synthesis while the tube which received labeled dATP for first strand synthesis received 1 microliter of water. Each tube then received 0.6 microliter of 7 U/microliter E. coli DNA ligase (Boehringer-Mannheim, Indianapolis, Ind.), 3.1 microliters of 8 U/microliter E. coli DNA polymerase I (Amersham), and 1 microliter 2 U/microliter of RNase H (Gibco-BRL). The reactions were incubated at 16° C. for 2 hours. After incubation, 2 microliters from each reaction was used to determine TCA precipitable counts and total counts in the reaction, and 2 microliters from each reaction was analyzed by gel electrophoresis. To the remainder of each sample, 2 microliters of 2.5 micrograms/microliter oyster glycogen, 5 microliters of 0.5 EDTA and 200 microliters of 10 mM Tris-HCl pH 7.4, 1 mM EDTA were added. The samples were phenol-chloroform extracted and isopropanol precipitated. After centrifugation the pellets were washed with 100 microliters of 80% ethanol and air dried. The yield of double stranded cDNA in each of the reactions was approximately 2.5 micrograms.

Mung bean nuclease treatment was used to clip the single-stranded DNA of the hair-pin. Each cDNA pellet was resuspended in 15 microliters of water and 2.5 microliters of 10×mung bean buffer (0.3 M NaAc pH 4.6, 3 M NaCl, and 10 mM ZnSO₄), 2.5 microliters of 10 mM DTT, 2.5 microliters of 50% glycerol, and 2.5-microliters of 10 U/microliter mung bean nuclease (New England Biolabs, Beverly, Mass.) were added to each tube. The reactions were i incubated at 30° C. for 30 minutes and 75 microliters of 10 mM Tris-HCl pH 7.4 and 1 mM EDTA was added to each tube. Two-microliter aliquots were analyzed by alkaline agarose gel analysis. One hundred microliters of 1 M Tris-HCl pH 7.4 was added to each tube and the samples were phenol-chloroform extracted twice. The DNA was isopropanol precipitated and pelleted by centrifugation. After centrifugation, the DNA pellet was washed with 80% ethanol and air dried. The yield was approximately 2 micrograms of DNA from each of the two reactions.

The cDNA ends were blunted by treatment with T4 DNA polymerase. DNA from the two samples were combined after resuspension in a total volume of 24 μl of water. Four microliters of 10× T4 buffer (330 mM Tris-acetate pH 7.9, 670 mM KAc, 100 mM MgAc, and 1 mg/ml gelatin), 4 microliters of 1 mM dNTP, 4 microliters 50 mM DTT, and 4 microliters of 1 U/microliter T4 DNA polymerase (Boehringer-Mannheim) were added to the DNA. The samples were incubated at 15° C. for 1 hour. After incubation, 160 microliters of 10 mM Tris-HCl pH 7.4, 1 mM EDTA was added, and the sample was phenol-chloroform extracted. The DNA was isopropanol precipitated and pelleted by centrifugation. After centrifugation the DNA was washed with 80% ethanol and air dried.

After resuspension of the DNA in 6.5 microliters water, Eco RI adapters were added to the blunted DNA. One microliter of 1 microggram/microliter Eco RI adapter (Invitrogen, San Diego, Calif. Cat. # N409-20), 1 microliter of 10× ligase buffer (0.5 M Tris pH 7.8 and 50 mM MgCl₂), 0.5 microliter of 10 mM ATP, 0.5 microliter of 100 mM DTT, and 1 microliter of 1 U/microliter T4 DNA ligase (Boehringer-Mannheim) were added to the DNA. After the sample was incubated overnight at room temperature, the ligase was heat denatured at 65° C. for 15 minutes.

The Sst I cloning site encoded by the first strand primer was exposed by digestion with Sst I endonuclease. Thirty-three microliters of water, 5 microliters of 10× Sst I buffer (0.5 M Tris pH 8.0, 0.1 M MgCl₂, and 0.5 M NaCl), and 2 microliters of 5 U/microliter Sst I were added to the DNA, and the samples were incubated at 37° C. for 2 hours. One hundred and fifty microliters of 10 mM Tris-HCl pH 7.4, 1 mM EDTA was added, the sample was phenol-chloroform extracted, and the DNA was isopropanol precipitated.

The cDNA was chromatographed on a Sepharose CL 2B (Pharmacia LKB Biotechnology) column to size-select the cDNA and to remove free adapters. A 1.1 ml column of Sepharose CL 2B was poured into a 1 ml plastic disposable pipet and the column was washed with 50 column volumes of buffer (10 mM Tris pH 7.4 and 1 mM EDTA). The sample was applied, one-drop fractions were collected, and the DNA in the void volume was pooled. The fractionated DNA was isopropanol precipitated. After centrifugation the DNA was washed with 80% ethanol and air dried.

A Fusarium oxysporum cDNA library was established by ligating the cDNA to the vector pYcDE8′ (cf. WO 90/10698) which had been digested with Eco RI and Sst I. Three hundred and ninety nanograms of vector was ligated to 400 ng of cDNA in a 80 microliter ligation reaction containing 8 microliters of 10× ligase buffer, 4 microliters of 10 mM ATP, 4 microliters 200 mM DTT, and 1 unit of T4 DNA ligase (Boehringer-Mannheim. After overnight incubation at room temperature, 5 micrograms of oyster glycogen and 120 microliters of 10 mM Tris-HCl and 1 mM EDTA were added and the sample was phenol-chloroform extracted. The DNA was ethanol precipitated, centrifuged, and the DNA pellet washed with 80% ethanol. After air drying, the DNA was resuspended in 3 microliters of water. Thirty seven microliters of electroporation competent DH10B cells (Gibco-BRL) was added to the DNA, and electroporation was completed with a Bio-Rad Gene Pulser (Model #1652076) and Bio-Rad Pulse Controller (Model #1652098) electroporation unit (Bio-Rad Laboratories, Richmond, Calif.). Four milliliters of SOC (Hanahan, J. Mol. Biol. 166 (1983), 557-580) was added to the electroporated cells, and 400 microliters of the cell suspension was spread on each of ten 150 mm LB amipicillin plates. After an overnight incubation, 10 ml of LB amp media was added to each plate, and the cells were scraped into the media. Glycerol stocks and plasmid preparations were made from each plate. The library background (vector without insert) was established at approximately 1% by ligating the vector without insert and titering the number of clones after electroporation.

To isolate full length cDNA clones of the 43 kD homologue a library of 1,100,000 clones was plated out onto 150 mm LB plates with 100 micrograms/ml ampicillin. One hundred thousand clones were plated out from glycerol stocks onto each of 10 plates and 20,000 clones were plated out on each of 5 plates. Lifts were taken in duplicate as described above. Prehydridization, hybridization and washing were also carried out as described above. Two end labeled 42-mer oligonucleotides, ZC3709 and ZC3710 (which are specific for the 43 kD homologue), were used in the hybridization. Filters were washed once for 20 minutes with TMACL at 77° C. Twenty two spots showing up on duplicate filters were found. Corresponding areas on the plates were picked with the large end of a pipet into 1 ml of 1× PCR buffer. These isolated analyses by PCR were with 2 sets of oligonucleotides for each isolate. One set contained the two 43 kD specific oligonucleotides used as hybridization probes and the other contained one 43 kD specific oligonucleotide, ZC3709, and one vector specific oligonucleotide, ZC3634. PCR was conducted as before by Perkin Elmer Cetus directions. Twenty picomoles of each primer and 5 microliters of the cell suspension were used in each reaction of 50 microliters. After an initial 1 minute 30 second denaturation at 94° C. 30 cycles of 1 minute at 94° C. and 2 minutes at 72° C. were employed, with a final extension time of 10 minutes at 72° C. Results showed 17 of the 22 to contain the 2 43 kD specific oligonucleotide recognition sites. The remaining 5 clones contained one of the 2 sites, ZC3709, but were shown by PCR with the vector specific primer to be truncated and not long enough to contain the other site. The 9 longest clones were chosen for single colony isolation through another level of screening. Five 10 fold dilutions of each were plated out and processed as described above for the first set of lifts. All of the nine had signals on autoradiograms of the second level of screening. Colonies were fairly congested so a few separate colonies in the area of the radioactive signal were single colony isolated on 150 mm LB plates with 70 μg/ml ampicillin. These were tested by PCR for homologues to the ˜43 kD endoglucanase with the oligonucleotides ZC3709 and ZC3710 as described for the first level of screening except that colonies were picked by toothpick into 25 microliters of mastermix. Bands of the expected size were obtained for 7 of the 9 clones. Cultures of these were started in 20 ml of Terrific Broth with 150 μg/ml ampicillin. DNA was isolated by alkaline lysis and PEG precipitation as above.

DNA Sequence Analysis

The cDNAs were sequenced in the yeast expression vector pYCDE8′. The dideoxy chain termination method (F. Sanger et al., Proc. Natl. Acad. Sci. USA 74, 1977, pp. 5463-5467) using @35-S dATP from New England Nuclear (cf. M. D. Biggin et al., Proc. Natl. Acad. Sci. USA 80, 1983, pp. 3963-3965) was used for all sequencing reactions. The reactions were catalysed by modified t7 DNA polymerase from Pharmacia (cf. S. Tabor and C. C. Richardson, Proc. Natl. Acad. Sci. USA 84, 1987, pp. 4767-4771) and were primed with an oligonucleotide complementary to the ADH1 promoter (ZC996: ATT GTT CTC CTT CCC TTT CTT), complementary to the CYC1 terminator (ZC3635: TGT ACG CAT GTA ACA TTA (SEQ ID NO: 27)) or with oligonucleotides complementary to the DNA of interest. Double stranded templates were denatured with NaOH (E. Y. Chen and P. H. Seeburg, DNA 4, 1985, pp. 165-170) prior to hybridizing with a sequencing oligonucleotide. Oligonucleotides were synthesized on an Applied Biosystems Model 380A DNA synthesizer. The oligonucleotides used for the sequencing reactions are listed in the sequencing oligonucleotide table below:

TABLE 1

Oligonucleotides for 43 kD homologue PCR:

| | | |
|---|---|---|
| ZC3485 | TGG GA(C/T) TG(C/T) TG(C/T) AA(A/G) CC | (SEQ ID NO:17) |
| ZC3486 | AGG GAG ACC GGA ATT CTG GGA (C/T)TG (C/T)TG (C/T) | (SEQ ID NO:18) |
| | AA(A/G) CC | |
| ZC3556 | CC(A/C/G/T) GG(A/C/G/T) GG(A/C/G/T) GG(A/C/G/T) | (SEQ ID NO:19) |
| | GT(A/C/G/T) GG | |
| ZC3557 | AGG GAG ACC GGA ATT CCC (A/C/G/T)GG (A/C/G/T)GG | (SEQ ID NO:20) |
| | (A/C/G/T)GG (A/C/G/T)GT (A/C/G/T)GG | |
| ZC3558 | AC(A/C/G/T) A(C/T)C AT(A/C/G/T) (G/T)T/C/T) TT(A/C/G/T) | (SEQ ID NO:21) |
| | CC | |
| ZC3559 | GAC AGA GCA CAG AAT TCA C(A/C/G/T)A (C/T)CA | (SEQ ID NO:22) |
| | T(A/C/G/T) (G/T) T(C/T)T T(A/C/G/T)C C | |
| ZC3560 | (A/C/G/T)GG (A/G)TT (A/G)TC (A/C/G/T)GC | (SEQ ID NO:23) |
| | (A/C/G/T) (G/T) (C/T) (C/T)T(C/T) (A/G)AA CCA | |
| ZC3561 | GAC AGA GCA CAG AAT TC(A/C/G/T) GG(A/G) TT(A/G) | (SEQ ID NO:24) |
| | TC(A/C/G/T) GC(A/C/G/T) (G/T) (C/T) (C/T) T(C/T) (A/G) | |
| | AAC CA | |

Oligonucleotides for 43 kD homologue cloning:

| | | |
|---|---|---|
| ZC3709 | GGG GTA GCT ATC ACA TTC GCT TCG GGA GGA GAT ACC GCC | (SEQ ID NO:25) |
| | GTA | |
| ZC3710 | CTT CTT GCT CTT GGA GCG GAA AGG CTG CTG TCA ACG CCC | (SEQ ID NO:26) |
| | CTG | |

pYCDE8′ vector oligonucleotides:

| | | | |
|---|---|---|---|
| ZC3635 | TGT ACG CAT GTA ACA TTA | (SEQ ID NO:27) | CYC 1 |
| | | | terminator |
| ZC3634 | CTG CAC AAT ATT TCA AGC | (SEQ ID NO:28) | ADH 1 promoter |

43 kD homologue specific sequencing primers:

| | | |
|---|---|---|
| ZC3709 | GGG GTA GCT ATC ACA TTC GCT TCG GGA GGA GAT ACC GCC GTA | (SEQ ID NO:29) |
| ZC3710 | CTT CTT GCT CTT GGA GCG GAA AGG CTG CTG TCA ACG CCC CTG | (SEQ ID NO:30) |
| ZC3870 | AGC TTC TCA AGG ACG GTT | (SEQ ID NO:31) |
| ZC3881 | AAC AAG GOT CGA ACA CTT | (SEQ ID NO:32) |
| ZC3882 | CCA GAA GAC CAA GGA TT | (SEQ ID NO:33) |

### Example 4

Color Clarification Test

The Humicola ˜43 kD endoglucanase (a mixture of 30 purification runs) was compared in a color clarification test with the H. insolens cellulase preparation described in U.S. Pat. No. 4,435,307, Example 6.

Old worn black cotton swatches are used as the test material. The clarification test is made in a Terg-O-tometer making three repeated washes. Between each wash the swatches are dried overnight.

Conditions:

2 g/l of liquid detergent at 40° C. for 30 min. and a water hardness of 9° dH. The swatch size is 10×15 cm, and there are two swatches in each beaker.

The composition of the detergent was as follows:

10% anionic surfactant (Nansa 1169/p)

15% non-ionic surfactant (Berol 160)

10% ethanol

5% triethanol amine

60% water

pH adjusted to 8.0 with HCl.

Dosage:

The two enzymes are dosed in 63 and 125 CMC-endoase units/l.

Results:

The results were evaluted by a panel of 22 persons who rated the swatches on a scale from 1 to 7 points. The higher the score, the more color clarification obtained.

| Enzyme | CMC-endoase/l | Protein mg/l | PSU* |
|---|---|---|---|
| No enzyme | | | 1.4 ± 1.0 |
| H. insolens | 63 | 14 | 5.8 ± 1.0 |
| cellulase | 125 | 28 | 6.1 ± 1.0 |
| mixture | | | |
| Invention | 63 | 0.4 | 4.6 ± 0.9 |
| | 125 | 0.8 | 6.2 ± 0.8 |

*PSU = Panel Score Units

The ˜43 kD endoglucanase is shown to have an about 30 times better performance than the prior art H. insolens cellulase mixture and an about 6 times better performance than the cellulase preparation according to WO 89/09259.

### Example 5

Stability of the Humicola ˜43 kD Endoglucanase in the Presence of Proteases

The storage stability of the ˜43 kD endoglucanase in liquid detergent in the presence of different proteases was determined under the following conditions:

Enzymes

˜43 kD endoglucanase of the invention

Glu/Asp specific B. licheniformis serine protease

Trypsin-like Fusarium sp. DSM 2672 protease

B. lentus serine protease

Subtilisin Novo

Detergent

US commercial liquid detergent not containing any opacifier, perfume or enzymes (apart from those added in the experiment). 1% (w/w) boric acid as enzyme stabilizer.

Dosage

| | |
|---|---|
| Endoglucanase: | 12 CMCU/g of detergent |
| Proteases: | 0.2 mg/g of detergent |

Incubation

7 days at 35° C.

Residual Activity

The residual activity of the endoglucanase after 7 days of incubation with the respective proteases was determined in terms of its CMCase activity (CMCU).

The CMCase activity was determined as follows:

A substrate solution of 30 g/l CMC (Hercules 7 LFD) in deionized water was prepared. The enzyme sample to be determined was dissolved in 0.01 M phosphate buffer, pH 7.5. 1.0 ml of the enzyme solution and 2.0 ml of a 0.1 M phosphate buffer, pH 7.5, were mixed in a test tube, and an enzyme reaction was initiated by adding 1.0 ml of the substrate solution to the test tube. The mixture was incubated at 40° C. for 20 minutes, after which the reaction was stopped by adding 2.0 ml of 0.125 M trisodium phosphate.12H₂O. A blind sample was prepared without incubation.

2.0 ml of a ferricyanide solution (1.60 g of potassium ferricyanide and 14.0 g of trisodium phosphate.12H₂O in 1 l of deionized water) was added to a test sample as well as to a blind immediately followed by immersion in boiling water and incubation for 10 minutes. After incubation, the samples were cooled with tap water. The absorbance at 420 nm was measured, and a standard curve was prepared with glucose solution.

One CMCase unit (CMCU) is defined as the amount of enzyme which, under the conditions specified above, forms an amount of reducing carbohydrates corresponding to 1 micromole of glucose per minute.

Results

The storage stability of the endoglucanase of the invention was determined as its residual activity (in CMCU %) under the conditions indicated above.

Residual Activity (%)

| Protease | + boric acid | − boric acid |
|---|---|---|
| Glu/Asp specific | 105 | 93 |
| Trypsin-like | 77 | 63 |
| B. lentus serine | 57 | 24 |
| Subtilisin Novo | 63 | 55 |

These results indicate that the storage stability in liquid detergent of the endoglucanase of the invention is improved when a protease with a higher degree of specificity than Savinase is included in the detergent composition.

### Example 6

Use of Humicola ˜43 kD Endoglucanase to Provide a Localized Variation in Color of Denim Fabric

Denim jeans were subjected to treatment with the ˜43 kD endoglucanase in a 12 kg “Wascator” FL120 wash extractor with a view to imparting a localized variation in the surface color of the jeans approximating a “stonewashed” appearance.

Four pairs of jeans were used per machine load. The experimental conditions were as follows.

Desizing

40 l water

100 ml B. amyloliquefaciens amylase*, 120 L

70 g KH₂PO₄

30 g Na₂HPO₄

55° C.

10 minutes

pH 6.8

*available from Novo Nordisk A/S.

The desizing process was followed by draining.

Abrasion

40 l water

120 g H. insolens cellulase mixture or

×g ˜43 kD endoglucanase

70 g KH₂PO₄

30 g Na₂HPO₄

55° C.

75 minutes

pH 6.6

The abrasion process was followed by draining, rinsing, after-washing and rinsing.

The results were evaluated by judging the visual appearance of the jeans.

Different dosages of ˜43 kD endoglucanase were used to obtain an abrasion level which was equivalent to that obtained with 120 g H. insolens cellulase mixture. Such an equivalent level was obtained with 1.0-1.25 g of ˜43 kD endoglucanase. Measurements of the tear strength of the treated garments showed no significant difference between the two enzyme treatments.

### Example 7

Use of Humicola ˜43 kD Endoglucanase to Remove Fuzz from Fabric Surface

Woven, 100% cotton fabric was treated with the ˜43 kD endoglucanase in a 12 kg “Wascator” FL120 wash extractor with a view to investigating the ability of the enzyme to impart a greater degree of softness to new fabric.

The experimental conditions were as follows.

Fabric

Woven, 100% cotton fabric obtained from Nordisk Textil, bleached (NT2116-b) or unbleached (NT2116-ub). 400 g of fabric were used per machine load.

Desizing

40 l water

200 ml B. amyloliquefaciens amylase, 120 L

60 g KH₂PO₄

20 g Na₂HPO₄

60° C.

10 minutes

pH 6.4

The desizing process was followed by draining.

Main Wash

40 l water

0-600 g H. insolens cellulase mixture or

×g ˜43 kD endoglucanase

60 g KH₂PO₄

40 g Na₂HPO₄

60° C.

60 minutes

pH 6.7

The abrasion step was followed by draining.

Afterwash

40 l water

40 g Na₂CO₃

10 g Berol 08

80° C.

15 minutes

pH 10.1

The afterwash was followed rinsing.

Three different concentrations of the ˜43 kD endoglucanase were added in the main wash.

The weight loss of the fabric samples was measured before and after treatment. The weight loss is expressed in % and is related to the desized fabric.

Fabric thickness was measured by means of a thickness measurer L&W, type 22/1.2 swatches of the fabric (10×6 cm) were measured, and 5 measurements in μm were recorded for each swatch. The swatch was measured at a pressure of 98.07 kPa. The retained thickness is expressed in % in relation to the desized fabric.

Fabric strength was measured by means of a tearing tester (Elmendorf 09). 6 swatches (10×6 cm) were cut in the warp direction and 6 swatches (10×6 cm) in the weft direction. The tear strength was measured in mN in accordance with ASTM D 1424. The fabric strength of the enzyme-treated fabric is expressed in % in relation to the desized fabric.

Fabric stiffness was measured by means of a King Fabric Stiffness Tester. 4 swatches (10×20 cm; 10 cm in the warp direction) are cut from the fabric, and each swatch is folded back to back (10×10 cm) and placed on a table provided with an open ring in the middle. A piston pushes the fabric through the ring using a certain power expressed in grammes. The determination is made according to the ASTM D 4032 Circular Bend Test Method. Retained fabric stiffness is expressed in % in relation to the desized fabric.

The results of these tests appear from the following table:

| Enzyme | Weight | Retained | Retained | Retained |
|---|---|---|---|---|
| Dosage | Loss | Thickness | Strength | Stiffness |
| EUG/l | % | % | % | % |
| 0 | 0 | 100 | 100 | 100 |
| 13 | 4.0 | 95.3 | 85.4 | 88.6 |
| 50 | 5.1 | 94.5 | 73.3 | 85.0 |
| 150 | 7.7 | 91.9 | 70.7 | 79.3 |

### Example 8

Use of Humicola ˜43 kD Endoglucanase for the Treatment of Paper Pulp

The ˜43 kD endoglucanase was used for the treatment of several types of paper pulp with a view to investigating the effect of the enzyme on pulp drainage.

The experimental conditions were as follows.

Pulps

1. Waste paper mixture: composed of 33% newsprint, 33% magazines and 33% computer paper. With or without deinking chemicals (WPC or WP, respectively)

2. Recycled cardboard containers (RCC).

3. Bleached kraft: made from pine (BK).

4. Unbleached thermomechanical: made from fir (TMP).

Determination of Cellulase Activity (CEVU)

A substrate solution containing 33.3 g/l CMC (Hercules 7 LFD) in Tris-buffer, pH 9.0, is prepared. The enzyme sample to be determined is dissolved in the same buffer. 10 ml substrate solution and 0.5 ml enzyme solution are mixed and transferred to a viscosimeter (Haake VT 181, NV sensor, 181 rpm) thermostated at 40° C. One Cellulase Viscosity Unit (CEVU) is defined in Novo Nordisk Analytical Method No. AF 253 (available from Novo Nordisk).

Determination of Pulp Drainage (Schopper-Riegler)

The Schopper-Riegler number (SR) is determined according to ISO standard 5267 (part 1) on a homogeneous pulp with a consistency of 2 g/l. A known volume of pulp is allowed to drain through a metal sieve into a funnel. The funnel is provided with an axial hole and a side hole. The volume of filtrate that passes through the side hole is measured in a vessel graduated in Schopper-Riegler units.

Enzymatic Treatment

A preparation of the ˜43 kD endoglucanase was diluted to 7 CEVU/ml and added to each of the pulps indicated above (50 g DS, consistency 3%). The enzyme dose was 2400 CEVU/kg dry pulp. The enzymatic treatment was conducted at a pH of 7.5 and at 40° C. with gentle stirring for 60 minutes. A sample was taken after 30 minutes to monitor the progression of the reaction. After 60 minutes, the pulp was diluted to a consistency of 0.5% with cold water (+4° C.) in order to stop the reaction.

Drainage of the wet pulp was determined as described above and assigned Schopper-Riegler (SR) values. The drainage time (DT) under vacuum was also determined.

The results are summarized in the following table.

Waste paper + chemicals

| | Control | Enzyme |
|---|---|---|
| SR (3%) | 61 | 55 |
| Drainage time (s) | 18.2 | 17 |
| 150 g/m² | | |
| Mass g/m² | 65.6 | 66.4 |
| Vol cm³/g | 1.65 | 1.66 |
| Breaking Length, m | 3650 | 3970 |
| Burst Index | 2.19 | 2.47 |

Waste paper

| | Control | Enzyme |
|---|---|---|
| SR (3%) | 59 | 51 |
| Drainage time (s) | 18.2 | 12.7 |
| 150 g/m² | | |
| Mass g/m² | 68.0 | 67.9 |
| Vol cm³/g | 1.68 | 1.64 |
| Breaking Length, m | 3810 | 3790 |
| Burst Index | 2.25 | 2.33 |

Recycled Cardboard
Containers

| | Control | Enzyme |
|---|---|---|
| SR (3%) | 45 | 33 |
| Drainage time (s) | 6.8 | 5.3 |
| 150 g/m² | | |
| Mass g/m² | 70.2 | 67.3 |
| Vol cm³/g | 1.91 | 1.99 |
| Breaking Length, m | 3640 | 3530 |
| Burst Index | 2.25 | 2.22 |

Kraft

| | Control | Enzyme |
|---|---|---|
| SR (3%) | 42 | 31 |
| Drainage time (s) | 10.7 | 6 |
| 150 g/m² | | |
| Mass g/m² | 67.5 | 69.1 |
| Vol cm³/g | 1.44 | 1.42 |
| Breaking Length, m | 7010 | 7190 |
| Burst Index | 5.14 | 4.96 |

TMP

| | Control | Enzyme |
|---|---|---|
| SR (3%) | 68 | 60 |
| Drainage time (s) | 13.8 | 11.3 |
| 150 g/m² | | |
| Mass g/m² | 68.7 | 70.2 |
| Vol cm³/g | 2.13 | 2.04 |
| Breaking Length, m | 3630 | 3620 |
| Burst Index | 1.95 | 1.91 |

Table 3: Results of the drainage and strength measurements.

Control experiments. Same conditions as the enzyme treatment

It appears from the table that the ˜43 kD endoglucanase treatment causes a significant decrease in SR values and significantly improves drainage of pulps used in papermaking.

Paper sheets were made from the various pulps on a Rapid then device and measured for strength according to different parameters (including breaking length). No decrease in strength properties due to enzyme action was observed.

1. A cellulase preparation consisting essentially of a homogenous endoglucanase component which is immunoreactive with an antibody raised against a highly purified ˜43 kD endoglucanase derived from Humicola insolens, DSM 1800, or which is homologous to said ˜43 kD endoglucanase.

2. A cellulase preparation according to claim 1, wherein the endoglucanase component has an endoglucanase activity of at least 50 CMC-endoase units/mg of protein.

3. A cellulase preparation according to claim 2, wherein the endoglucanase component has an endoglucanase activity of at least 60 CMC-endoase units/mg of total protein, in particular at least 90 CMC-endoase units/mg of total protein, and preferably at least 100 CMC-endoase units/mg of total protein.

4. A cellulase preparation according to claim 1, wherein the endoglucanase component has essentially no cellobiohydrolase activity.

5. A cellulase preparation according to any of claims 1-4, wherein the endoglucanase component has an isoelectric point of about 5.1.

6. An enzyme exhibiting endoglucanase activity, which enzyme has the amino acid sequence shown in SEQ ID NO: 2, or a homologue thereof exhibiting endoglucanase activity.

7. An endoglucanase enzyme according to claim 6 which is producible by a species of Humicola, e.g. Humicola insolens.

8. An enzyme exhibiting endoglucanase activity, which enzyme has the amino acid sequence shown in SEQ ID NO: 4, or a homologue thereof exhibiting endoglucanase activity.

9. An endoglucanase enzyme according to claim 8 which is producible by a species of Fusarium, e.g. Fusarium oxysporum.

10. A DNA construct comprising a DNA sequence encoding an endoglucanase enzyme as claimed in any of claims 6-9.

11. A DNA construct according to claim 10, wherein the DNA sequence is as shown in SEQ ID NO: 1 or 3 or a modification thereof.

12. An expression vector which carries an inserted DNA sequence according to claim 10 or 11.

13. A cell which is transformed with a DNA construct according to claim 10 or 11 or with an expression vector according to claim 12.

14. A cell according to claim 13 which is a fungal cell, e.g. belonging to a strain of Trichoderma or Aspergillus, in particular Aspergillus oryzae or Aspergillus niger, or a yeast cell, e.g. belonging to a strain of Hansenula or Saccharomyces, e.g. Saccharomyces cerevisiae.

15. A process for producing an endoglucanase enzyme as defined in any of claims 6-9, the process comprising culturing a cell according to claim 13 or 14 in a suitable culture medium under conditions permitting the expression of the endoglucanase enzyme, and recovering the endoglucanase enzyme from the culture.

16. A detergent additive containing a cellulase preparation according to any of claims 1-5 or an endoglucanase enzyme according to any of claims 6-9, preferably in the form of a non-dusting granulate, stabilized liquid or protected enzyme.

17. A detergent additive according to claim 16 which contains 1-500, preferably 5-250, most preferably 10-100, mg of enzyme protein per gram of the additive.

18. A detergent additive according to claim 16 which additionally comprises another enzyme such as a protease, lipase, peroxidase and/or amylase.

19. A detergent additive according to claim 18, wherein the protease is one which has a higher degree of specificity than Bacillus lentus serine protease.

20. A detergent additive according to claim 19, wherein the protease is subtilisin Novo or a variant thereof, a protease derivable from Nocardia dassonvillei NRRL 18133, a serine protease specific for glutamic and aspartic acid, producible by Bacillus licheniformis, or a trypsin-like protease producible by Fusarium sp. DSM 2672.

21. A detergent composition comprising a cellulase preparation according to any of claims 1-5 or an endoglucanase enzyme according to any of claims 6-9.

22. A detergent composition according to claim 21, which additionally comprises another enzyme such as a protease, lipase, peroxidase and/or amylase.

23. A detergent composition according to claim 22, wherein the protease is one which has a higher degree of specificity than Bacillus lentus serine protease.

24. A detergent composition according to claim 23, wherein the protease is subtilisin Novo or a variant thereof, a protease derivable from Nocardia dassonvillei NRRL 18133, a serine protease specific for glutamic and aspartic acid, producible by Bacillus licheniformis, or a trypsin-like protease producible by Fusarium sp. DSM 2672.

25. A detergent composition according to claim 21, wherein the cellulase preparation or endoglucanase enzyme is present in a concentration corresponding to 0.01-100, preferably 0.05-60, and most preferably 0.1-20, mg of enzyme protein per liter washing solution.

26. A detergent composition comprising a detergent additive according to any of claims 16-20.

27. A method of reducing the rate at which cellulose-containing fabrics become harsh or of reducing the harshness of cellulose-containing fabrics, the method comprising treating cellulose-containing fabrics with a cellulase preparation according to any of claims 1-5 or an endoglucanase enzyme according to any of claims 6-9.

28. A method of providing color clarification of colored cellulose-containing fabrics, the method comprising treating colored cotton-containing fabrics with a cellulase preparation according to any of claims 1-5 or an endoglucanase enzyme according to any of claims 6-9.

29. A method of providing a localized variation in color of colored cellulose-containing fabrics, the method comprising treating colored cotton-containing fabrics with a cellulase preparation according to any of claims 1-5 or an endoglucanase enzyme according to any of claims 6-9.

30. A method according to any of claims 27, 28 or 29, wherein the treatment of the fabrics with the cellulase preparation is carried out during soaking, washing or rinsing of the fabrics.

31. A method of improving the drainage properties of pulp, the method comprising treating paper pulp with a cellulase preparation according to any of claims 1-5 or an endoglucanase enzyme according to any of claims 6-9.

32. An isolated enzyme exhibiting endoglucanase activity, which is encoded by a sequence that hybridizes to the sequence of SEQ ID NO:1 at 40° C. in 20% formamide-50 mM sodium phosphate pH 6.8.

33. The enzyme of claim 32, which is derived from Humicola.

34. The enzyme of claim 33, which is derived from Humicola insolens.

35. A detergent composition, comprising an enzyme of claim 32 and a surfactant.

36. A method of reducing the rate at which cellulose-containing fabrics become harsh or of reducing the harshness of cellulose-containing fabrics, comprising contacting the cellulose-containing fabrics with an enzyme of claim 32.

37. A method of providing color clarification of colored cellulose-containing fabrics, comprising contacting the colored cellulose-containing fabrics with an enzyme of claim 32.

38. A method of providing a localized variation in color of colored cellulose-containing fabrics, comprising treating the colored cotton-containing fabrics with an enzyme of claim 32.

39. A method of improving the drainage properties of paper pulp, comprising treating the paper pulp with an enzyme of claim 32.

40. An isolated enzyme exhibiting endoglucanase activity, which is encoded by a sequence that hybridizes to the sequence of SEQ ID NO:3 at 40° C. in 20% formamide-50 mM sodium phosphate pH 6.8.

41. The enzyme of claim 40, wherein said sequence is derived from Fusarium.

42. The enzyme of claim 41, wherein said sequence is derived from Fusarium oxysporum.

43. A detergent composition, comprising the enzyme of claim 40 and a surfactant.

44. A method of reducing the rate at which cellulose-containing fabrics become harsh or of reducing the harshness of cellulose-containing fabrics, comprising contacting the cellulose-containing fabrics with an enzyme of claim 40.

45. A method of providing color clarification of colored cellulose-containing fabrics, comprising contacting the colored cellulose-containing fabrics with an enzyme of claim 40.

46. A method of providing a localized variation in color of colored cellulose-containing fabrics, comprising treating the colored cotton-containing fabrics with an enzyme of claim 40.

47. A method of improving the drainage properties of paper pulp, comprising treating the paper pulp with an enzyme of claim 40.
